(19) United States
(12) Patent Application Publication
(10) Pub. No.: US 2009/0111154 A1
(43) Pub. Date: Apr. 30, 2009

(54) BUTANOL PRODUCTION BY RECOMBINANT MICROORGANISMS

(75) Inventors: James C. Liao, Los Angeles, CA (US); Shota Atsumi, Los Angeles, CA (US); Mark P. Brynildsen, Newton, MA (US); Anthony F. Cann, Los Angeles, CA (US); Katherine J. Chou, Los Angeles, CA (US); Roa Pu Claire Shen, Los Angeles, CA (US); Kevin M. Smith, Beverly Hills, CA (US); Taizo Hanai, Higashi-Ku (JP); Michael R. Connor, Los Angeles, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, OAKLAND, CA (US)

(21) Appl. No.: 12/062,398

(22) Filed: Apr. 3, 2008

Related U.S. Application Data
(60) Provisional application No. 60/921,927, filed on Apr. 4, 2007.
(60) Provisional application No. 60/939,978, filed on May 24, 2007.

(51) Int. Cl. C12P 7/16; C12N 1/21; C12N 15/63

(52) U.S. Cl. 435/160; 435/252.33; 435/252.3; 435/252.35; 435/320.1

(57) ABSTRACT

Provided are microorganisms that catalyze the synthesis of biofuels from a suitable substrate such as glucose. Also provided are methods of generating such organisms and methods of synthesizing biofuels using such organisms. Provided are microorganisms comprising non-naturally occurring metabolic pathway for the production of higher alcohols.

### CROSS REFERENCE TO RELATED APPLICATIONS

This application claims priority to U.S. Provisional Application Ser. No. 60/921,927 filed Apr. 4, 2007, and to U.S. Provisional Application Ser. No. 60/939,978 filed May 24, 2007, the disclosures of which are incorporated herein by reference in their entirety.

### TECHNICAL FIELD

Metabolically-modified microorganisms and methods of producing such organisms are provided. Also provided are methods of producing biofuels by contacting a suitable substrate with a metabolically-modified microorganism and enzymatic preparations there from.

### BACKGROUND

Global energy and environmental issues have prompted increased efforts in synthesizing biofuels from renewable resources. Existing biofuels such as ethanol and butanol are common fermentation products of microorganisms. n-Butanol is generally preferred because of its hydrophobicity, lower vapor pressure, and higher energy content.

### SUMMARY

Provided herein are metabolically-modified microorganisms that include recombinant biochemical pathways useful for producing n-butanol via fermentation of a suitable substrate. Also provided are methods of producing biofuels using microorganisms described herein.

In one embodiment, a recombinant microorganism including a recombinant biochemical pathway to produce n-butanol from fermentation of a suitable carbon substrate is provided.

In one aspect, a recombinant microorganism provided herein includes elevated expression of a keto thiolase as compared to a parental microorganism. The recombinant microorganism produces a metabolite that includes acetoacetyl-CoA from a substrate that includes acetyl-CoA. The keto thiolase can be encoded by an atoB gene or homolog thereof, or a fadA gene or homolog thereof. The atoB gene or fadA gene can be derived from the genus Escherichia.

In another aspect, a recombinant microorganism provided herein includes elevated expression of an acetyl-CoA acetyltransferase as compared to a parental microorganism. The microorganism produces a metabolite that includes acetoacetyl-CoA from a substrate that includes acetyl-CoA. The acetyl-CoA acetyltransferase can be encoded by a thlA gene or homolog thereof. The thlA gene can be derived from the genus Clostridium.

In another aspect, a recombinant microorganism provided herein includes elevated expression of hydroxybutyryl-CoA dehydrogenase as compared to a parental microorganism. The recombinant microorganism produces a metabolite that includes a 3-hydroxybutyryl-CoA from a substrate that includes acetoacetyl-CoA. The hydroxybutyryl CoA dehydrogenase can be encoded by an hbd gene or homolog thereof. The hbd gene can be derived from various microorganisms including Clostridiuum acetobutylicum, Clostridium difficile, Dastricha ruminatium, Butyrivibrio fibrisolvens, Treponema phagedemes, Acidaminococcus fermentans, Clostridium kluyveri, Syntrophosphora bryanti, and Thermoanaerobacterium thermosaccharolyticum.

In another aspect, a recombinant microorganism provided herein includes elevated expression of crotonase as compared to a parental microorganism. The recombinant microorganism produces a metabolite that includes crotonyl-CoA from a substrate that includes 3-hydroxybutyryl-CoA. The crotonase can be encoded by a crt gene or homolog thereof. The crt gene can be derived from various microorganisms including Clostridium acetobutylicum, Butyrivibrio fibrisolvens, Thermoanaerobacterium thermosaccharolyticum, and Clostridium difficile.

In yet another aspect, a recombinant microorganism provided herein includes elevated expression of a crotonyl-CoA reductase as compared to a parental microorganism. The microorganism produces a metabolite that includes butyryl-CoA from a substrate that includes crotonyl-CoA. The crotonyl-CoA reductase can be encoded by a ccr gene or homolog thereof. The ccr gene can be derived from the genus Streptomyces.

In yet another aspect a recombinant microorganism provided herein includes elevated expression of a butyryl-CoA dehydrogenase as compared to a parental microorganism. The recombinant microorganism produces a metabolite that includes butyryl-CoA from a substrate that includes crotonyl-CoA. The butyryl-CoA dehydrogenase can be encoded by a bcd gene or homolog thereof. The bcd gene can be derived from Clostridium acetobutylicum, Mycobacterium tuberculosis, or Megasphaera elsdenii.

In yet another aspect a recombinant microorganism provided herein includes elevated expression of an alcohol dehydrogenase (ADH) as compared to a parental microorganism. The recombinant microorganism produces a metabolite that includes butanol from a substrate that includes butyryl-CoA. The alcohol dehydrogenase can be encoded by an aad gene or homolog thereof, or an adhE gene or homolog thereof. These enzymes are members of a class of enzymes that possess alcohol/aldehyde dehydrogenase activity. For example, the E. coli adhE enzyme converts acetyl-CoA to ethanol. The aad gene or adhE2 gene can be derived from Clostridium acetobutylicum.

In another embodiment, a recombinant microorganism including a recombinant biochemical pathway to produce n-butanol from fermentation of a suitable carbon substrate is provided. The recombinant biochemical pathway includes elevated expression of: a) a keto thiolase as compared to a parental microorganism or an acetyl-CoA acetyltransferase as compared to a parental microorganism; b) a hydroxybutyryl-CoA dehydrogenase as compared to a parental microorganism; c) a crotonase as compared to a parental microorganism; d) a crotonyl-CoA reductase as compared to a parental microorganism or a butyryl-CoA dehydrogenase as compared to a parental microorganism; and e) an alcohol dehydrogenase (ADH) as compared to a parental microorganism.

In yet another embodiment, a method of producing a recombinant microorganism that converts a suitable carbon substrate to n-butanol is provided. The method includes transforming a microorganism with one or more recombinant polynucleotides encoding polypeptides that include keto thiolase or acetyl-CoA acetyltransferase activity, hydroxybutyryl-CoA dehydrogenase activity, crotonase activity, crotonyl-CoA reductase or butyryl-CoA dehydrogenase, activity, and alcohol dehydrogenase activity.

In another embodiment, a method for producing n-butanol is provided. The method includes: a) providing a recombinant microorganism as provided herein; b) culturing the microorganism in the presence of a suitable carbon substrate and under conditions suitable for the conversion of the substrate to n-butanol; and c) detecting the production of n-butanol.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 depicts an exemplary pathway for the synthesis of n-butanol by a recombinant microorganism.

FIG. 2A depicts a map of plasmid pJCL4.

FIG. 2B depicts a map of plasmid pJCL31.

FIG. 3 depicts SEQ ID NO:66 and 68, a nucleic acid sequence of fadA and fadB, respectively.

FIG. 4 depicts a chromatogram of butanol production.

FIG. 5 depicts additional chromatograms of butanol production.

FIG. 6 depicts a chromatogram of a spike experiment.

FIG. 7 depicts mass spectrometry information.

FIG. 8 depicts SEQ ID NO:30, a nucleic acid sequence derived from an atoB gene encoding a polypeptide having keto thiolase activity.

FIG. 9 depicts SEQ ID NO:32, a nucleic acid sequence derived from a thlA gene encoding a polypeptide having acetyl-CoA acetyltransferase activity.

FIG. 10 depicts SEQ ID NO:34, a nucleic acid sequence derived from a crt gene encoding a polypeptide having crotonase activity.

FIG. 11 depicts SEQ ID NO:36, a nucleic acid sequence derived from a hbd gene encoding a polypeptide having hydroxybutyryl CoA dehydrogenase activity.

FIG. 12 depicts SEQ ID NO:38, a nucleic acid sequence derived from a bcd gene encoding a polypeptide having butyryl-CoA dehydrogenase activity.

FIG. 13 depicts SEQ ID NO:40, a nucleic acid sequence derived from an etfA gene encoding an ETF polypeptide.

FIG. 14 depicts SEQ ID NO:42, a nucleic acid sequence derived from an etfB gene encoding an ETF polypeptide.

FIG. 15 depicts SEQ ID NO:44, a nucleic acid sequence derived from a bcd gene encoding a polypeptide having butyryl-CoA dehydrogenase activity.

FIG. 16 depicts SEQ ID NO:46, a nucleic acid sequence derived from an etfA gene encoding an ETF polypeptide.

FIG. 17 depicts SEQ ID NO:48, a nucleic acid sequence derived from an etfB gene encoding an ETF polypeptide.

FIG. 18 depicts SEQ ID NO:50, a nucleic acid sequence derived from a ccr gene encoding a polypeptide having crotonyl CoA reductase activity.

FIG. 19 depicts SEQ ID NO:52, a nucleic acid sequence derived from a ccr gene encoding a polypeptide having crotonyl CoA reductase activity.

FIG. 20 depicts SEQ ID NO:54, a nucleic acid sequence derived from a ccr gene encoding a polypeptide having crotonyl CoA reductase activity.

FIG. 21 depicts SEQ ID NO:56, a nucleic acid sequence derived from a ccr gene encoding a polypeptide having crotonyl CoA reductase activity.

FIG. 22 depicts SEQ ID NO:58, a nucleic acid sequence derived from a ccr gene encoding a polypeptide having crotonyl CoA reductase activity.

FIG. 23 depicts SEQ ID NO:60, a nucleic acid sequence derived from a ccr gene encoding a polypeptide having crotonyl CoA reductase activity.

FIG. 24 depicts SEQ ID NO:62, a nucleic acid sequence derived from a ccr gene encoding a polypeptide having crotonyl CoA reductase activity.

FIG. 25 depicts SEQ ID NO:64, a nucleic acid sequence derived from a ccr gene encoding a polypeptide having alcohol dehydrogenase activity.

FIG. 26 provides a schematic representation of 1-butanol production in engineered E. coli. The exemplary 1-butanol production pathway includes 6 enzymatic steps from acetyl-CoA. AtoB, acetyl-CoA acetyltransferase; Thl, acetoacetyl-CoA thiolase; Hbd, 3-hydroxybutyryl-CoA dehydrogenase; Crt, crotonase; Bcd, butyryl-CoA dehydrogenase; Etf, electron transfer flavoprotein; AdhE2, aldehyde/alcohol dehydrogenase.

FIG. 27 depicts 1-Butanol production from engineered E. coli. Panel A provides exemplary results of an investigation of growth conditions and comparison of thl and atoB on production of 1-butanol. JCL191 and JCL198 were grown in an anaerobic condition (squares, ‘−’), an aerobic condition (triangles, ‘+’), and a semi-aerobic condition (circles, ‘S’) at 37° C. for 8-40 hr. Panel B provides the results of an evaluation of 1-butanol production using various enzymes for the reduction of crotonyl-CoA to butyryl-CoA. JCL187, JCL230 and JCL235 contain bcd-etfAB from C. acetobutylicum, ccr from S. coelicolor and bcd-etfAB from M. elsdenii, respectively. Cultures were grown semi-aerobically in shake flasks at 37° C. for 24 hr. Panel C provides a comparison of the effect of gene deletions on the production of 1-butanol in E. coli. Cells were grown semi-aerobically in with the addition of 0.1% casamino acids in shake flasks at 37° C. for 24 hr. “Δ” indicates gene deletion.

FIG. 28 shows a comparison of the effect of media on the production of 1-butanol in E. coli. Cells were grown semi-aerobically in M9 medium and TB medium supplemented with 2% glucose, 2% glycerol, or no additional carbon source at 37 1 C for 24 h.

Like reference symbols in the various drawings indicate like elements.

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

Butanol is hydrophobic and less volatile than ethanol. 1-Butanol has an energy density closer to gasoline. Butanol at 85 percent strength can be used in cars without any change to the engine (unlike ethanol) and it produces more power than ethanol and almost as much power as gasoline. Butanol is also used as a solvent in chemical and textile processes, organic synthesis and as a chemical intermediate. Butanol also is used as a component of hydraulic and brake fluids and as a base for perfumes.

The native producers of 1-butanol, such as Clostridium acetobutylicum, also produce byproducts such as acetone, ethanol, and butyrate as fermentation products. However, these microorganisms are relatively difficult to manipulate. Genetic manipulation tools for these organisms are not as efficient as those for user-friendly hosts such as E. coli and physiology and their metabolic regulation are much less understood, prohibiting rapid progress towards high-efficiency production.

The disclosure provides organisms comprising metabolically engineered biosynthetic pathways that utilize an organism's CoA pathway. Biofuel production utilizing the organism's CoA pathway offers several advantages. Not only does it avoid the difficulty of expressing a large set of foreign genes but it also minimizes the possible accumulation of toxic intermediates. Contrary to the butanol production pathway found in many species of Clostridium, the engineered amino acid biosynthetic routes for biofuel production circumvent the need to involve oxygen-sensitive enzymes and intermediates.

In one aspect, the disclosure provides a recombinant microorganism comprising elevated expression of at least one target enzyme as compared to a parental microorganism or encodes an enzyme not found in the parental organism. In another or further aspect, the microorganism comprises a reduction, disruption or knockout of at least one gene encoding an enzyme that competes with a metabolite necessary for the production of a desired higher alcohol product or which produces an unwanted product. The recombinant microorganism produces at least one metabolite involved in a biosynthetic pathway for the production of 1-butanol. In general, the recombinant microorganisms comprises at least one recombinant metabolic pathway that comprises a target enzyme and may further include a reduction in activity or expression of an enzyme in a competitive biosynthetic pathway. The pathway acts to modify a substrate or metabolic intermediate in the production of 1-butanol. The target enzyme is encoded by, and expressed from, a polynucleotide derived from a suitable biological source. In some embodiments, the polynucleotide comprises a gene derived from a bacterial or yeast source and recombinantly engineered into the microorganism of the disclosure.

As used herein, the term “metabolically engineered” or “metabolic engineering” involves rational pathway design and assembly of biosynthetic genes, genes associated with operons, and control elements of such polynucleotides, for the production of a desired metabolite, such as an acetoacetyl-CoA or higher alcohol, in a microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one aspect, where the polynucleotide is xenogenetic to the host organism, the polynucleotide can be codon optimized.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a metabolically engineered microorganism as described herein. A “biomass derived sugar” includes, but is not limited to, molecules such as glucose, sucrose, mannose, xylose, and arabinose. The term biomass derived sugar encompasses suitable carbon substrates ordinarily used by microorganisms, such as 6 carbon sugars, including, but not limited to, glucose, lactose, sorbose, fructose, idose, galactose and mannose in either D or L form, or a combination of 6 carbon sugars, such as glucose and fructose, and/or 6 carbon sugar acids including, but not limited to, 2-keto-L-gulonic acid, idonic acid (IA), gluconic acid (GA), 6-phosphogluconate, 2-keto-D-gluconic acid (2 KDG), 5-keto-D-gluconic acid, 2-ketogluconatephosphate, 2,5-diketo-L-gulonic acid, 2,3-L-diketogulonic acid, dehydroascorbic acid, erythorbic acid (EA) and D-mannonic acid.

The term “1-butanol” or “n-butanol” generally refers to a straight chain isomer with the alcohol functional group at the terminal carbon. The straight chain isomer with the alcohol at an internal carbon is sec-butanol or 2-butanol. The branched isomer with the alcohol at a terminal carbon is isobutanol, and the branched isomer with the alcohol at the internal carbon is tert-butanol.

Recombinant microorganisms provided herein can express a plurality of target enzymes involved in pathways for the production of 1-butanol from a suitable carbon substrate.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite. In an illustrative embodiment, the introduction of genetic material into a parental microorganism results in a new or modified ability to produce 1-butanol. The genetic material introduced into the parental microorganism contains gene(s), or parts of genes, coding for one or more of the enzymes involved in a biosynthetic pathway for the production of 1-butanol and may also include additional elements for the expression and/or regulation of expression of these genes, e.g. promoter sequences.

An engineered or modified microorganism can also include in the alternative or in addition to the introduction of a genetic material into a host or parental micoorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produced a new or greater quantities of an interacellular metabolite, improve the flux of a metabolite down a desired pathway, and/or reduce the production of undesirable by-products).

The disclosure demonstrates that the expression of one or more heterologous polynucleotide or over-expression of one or more heterologous polynucleotide encoding; (i) a polypeptide that catalyzes the production of acetoacetyl-coA from two molecules of acetyl-coA; (ii) a polypeptide that catalyzes the conversion of acetoacetyl-coA to 3-hydroxybutyryl-CoA; (iii) a polypeptide the catalyzes the conversion of 3-hydroxybutyryl-CoA to crotonyl-CoA; (iv) a polypeptide (or polypeptide combination) that catalyzes the reduction of crotonyl-CoA to butyryl-CoA; and (v) a polypeptide that preferentially catalyzes the conversion of butyryl-CoA to butyraldehyde and butyraldehyde to 1-butanol. For example, the disclosure demonstrates that with over-expression of the heterologous thl, hbd, crt, bcd, etfAB, and adhE2 genes in E. coli the production of 1-butanol can be obtained.

Microorganisms provided herein are modified to produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., acetyl-coA) in, or an end product (e.g., 1-butanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

Accordingly, the disclosure provides a recombinant microorganisms that produce 1-butanol and include the expression or elevated expression of target enzymes such as a acetyl-coA acetyl transferase (e.g., atoB), an acetoacetyl-coA thiolase (e.g., thl), a 3-hydroxybutryl-coA dehydrogenase (e.g., hbd), a crotonase (e.g., crt), a butyryl-CoA dehydrogeanse (e.g., bcd), and electron transfer flavoprotein (e.g., etf), and an aldehyde/alcohol dehydrognase (e.g., adhE2), or any combination thereof, as compared to a parental microorganism. In addition, the microorganism may include a disruption, deletion or knockout of expression of an alcohol/acetoaldehyde dehydrogenase the preferentially uses acetyl-coA as a substrate (e.g. adhE gene), as compared to a parental microorganism. Other disruptions, deletions or knockouts can include one or more genes encoding a polypeptide or protein selected from the group consisting of: (i) an enzyme that catalyzes the NADH-dependent conversion of pyruvate to D-lactate; (ii) an enzyme that promotes catalysis of fumarate and succinate interconversion; (iii) an oxygen transcription regulator; (iv) an enzyme catalyzes the conversion of acetyl-coA to acetyl-phosphate; and (v) an enzyme that catalyzes the conversion of pyruvate to acetyl)-coA and formate. In one aspect, the microorganism comprising a disruption, deletion or knockout of a combination of an alcohol/acetoaldehyde dehydrogenase and one or more of (i)-(iv) above, but not (v).

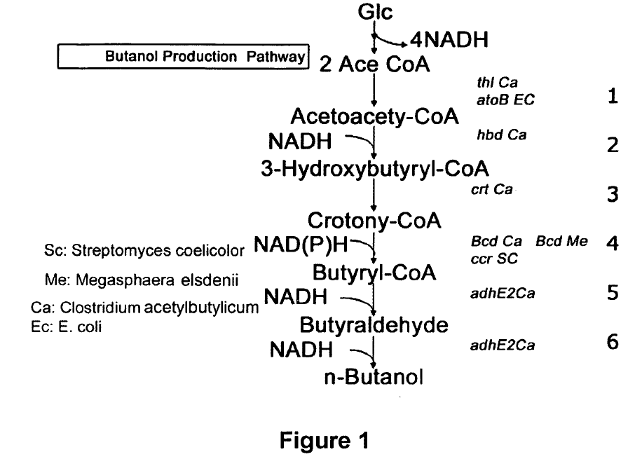

As depicted in FIG. 1, acetoacetyl-CoA can be produced by a recombinant microorganism metabolically engineered to express or over-express keto thiolase or acetyl-CoA acetyltransferase.

Additionally, 3-hydroxybutyryl-CoA can be produced by a recombinant microorganism metabolically engineered to express or over-express hydroxybutyryl CoA dehydrogenase and crotonyl-CoA can be produced by a recombinant microorganism metabolically engineered to express or over-express crotonase.

Further, the metabolite butyryl-CoA can be produced by a recombinant microorganism metabolically engineered to express or over-express crotonyl-CoA reductase or butyryl-CoA dehydrogenase.

The metabolites buteraldehyde and n-butanol can be produced by a recombinant microorganism metabolically engineered to express or over-express alcohol dehydrogenase (ADH).

Accordingly, a recombinant microorganism provided herein includes the elevated expression of at least one target enzyme, such as keto thiolase. In other aspects a recombinant microorganism can express a plurality of target enzymes involved in pathway to produce n-butanol from fermentation of a suitable carbon substrate. The plurality of enzymes can include keto thiolase, acetyl-CoA acetyltransferase, hydroxybutyryl CoA dehydrogenase, crotonase, crotonyl-CoA reductase, butyryl-CoA dehydrogenase, and alcohol dehydrogenase (ADH), or any combination thereof.

As previously noted, the target enzymes described throughout this disclosure generally produce metabolites. For example, a keto thiolase produces acetoacetyl-CoA from a substrate that includes acetyl-CoA. In addition, the target enzymes described throughout this disclosure are encoded by polynucleotide. For example, a keto thiolase can be encoded by an atoB gene, polynucleotide or homolog thereof, or an fadA gene, polynucleotide or homolog thereof. The atoB gene or fadA gene can be derived from any biologic source that provides a suitable nucleic acid sequence encoding a suitable enzyme. For example, atoB gene or fadA gene can be derived from E. coli or C. acetobutylicum.

In another aspect, a recombinant microorganism provided herein includes elevated expression of an acetyl-CoA acetyltransferase as compared to a parental microorganism. The microorganism produces a metabolite that includes acetoacetyl-CoA from a substrate that includes acetyl-CoA. The acetyl-CoA acetyltransferase can be encoded by a thlA gene, polynucleotide or homolog thereof. The thlA gene or polynucleotide can be derived from the genus Clostridium.

In another aspect, a recombinant microorganism provided herein includes elevated expression of a hydroxybutyryl CoA dehydrogenase as compared to a parental microorganism. The recombinant microorganism produces a metabolite that includes a 3-hydroxybutyryl-CoA from a substrate that includes acetoacetyl-CoA. The hydroxybutyryl CoA dehydrogenase can be encoded by a hbd gene, polynucleotide or homolog thereof. The hbd gene can be derived from various microorganisms including Clostridium acetobutylicum, Clostridium difficile, Dastricha ruminatium, Butyrivibrio fibrisolvens, Treponema phagedemes, Acidaminococcus fermentans, Clostridium kluyveri, Syntrophosphora bryanti, and Thermoanaerobacterium thermosaccharolyticum.

In another aspect, a recombinant microorganism provided herein includes elevated expression of crotonase as compared to a parental microorganism. The recombinant microorganism produces a metabolite that includes crotonyl-CoA from a substrate that includes 3-hydroxybutyryl-CoA. The crotonase can be encoded by a crt gene, polyncleotide or homolog thereof. The crt gene or polynucleotide can be derived from various microorganisms including Clostridium acetobutylicum, Butyrivibrio fibrisolvens, Thermoanaerobacterium thermosaccharolyticum, and Clostridium difficile.

In yet another aspect, a recombinant microorganism provided herein includes elevated expression of a crotonyl-CoA reductase as compared to a parental microorganism. The microorganism produces a metabolite that includes butyryl-CoA from a substrate that includes crotonyl-CoA. The crotonyl-CoA reductase can be encoded by a ccr gene, polynucleotide or homolog thereof. The ccr gene or polynucleotide can be derived from the genus Streptomyces.

In yet another aspect, a recombinant microorganism provided herein includes elevated expression of a butyryl-CoA dehydrogenase as compared to a parental microorganism. The recombinant microorganism produces a metabolite that includes butyryl-CoA from a substrate that includes crotonyl-CoA. The butyryl-CoA dehydrogenase can be encoded by a bcd gene, polynucleotide or homolog thereof. The bcd gene, polynucleotide can be derived from Clostridium acetobutylicum, Mycobacterium tuberculosis, or Megasphaera elsdenii.

In yet another aspect, a recombinant microorganism provided herein includes elevated expression of an alcohol dehydrogenase (ADH) as compared to a parental microorganism. The recombinant microorganism produces a metabolite that includes butanol from a substrate that includes butyryl-CoA. The alcohol dehydrogenase can be encoded by an aad gene, polynucleotide or homolog thereof, or an adhE gene, polynucleotide or homolog thereof. The aad gene or adhE gene or polynucleotide can be derived from Clostridium acetobutylicum.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a function enzyme activity using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The following table and the disclosure provides non-limiting examples of genes and homologs for each gene having polynucleotide and polypeptide sequences available to the skilled person in the art.

| | Exemplary | | |
|---|---|---|---|
| Enzyme | Gene(s) | 1-butanol | Exemplary Organism |
| Ethanol | adhE | − | E. coli |
| Dehydrogenase | | | |
| Lactate | ldhA | − | E. coli |
| Dehydrogenase | | | |
| Fumarate reductase | frdB, | − | E. coli |
| | frdC, or | | |
| | frdBC | | |
| Oxygen | fnr | − | E. coli |
| transcription | | | |
| regulator | | | |
| Phosphate | pta | − | E. coli |
| acetyltransferase | | | |
| Formate | pflB | − | E. coli |
| acetyltransferase | | | |
| acetyl-coA | atoB | + | C. acetobutylicum |
| acetyltransferase | | | |
| acetoacetyl-coA | thl, thlA, | + | E. coli, |
| thiolase | thlB | | C. acetobutylicum |
| 3-hydroxybutyryl- | hbd | + | C. acetobutylicum |
| CoA dehydrogenase | | | |
| crotonase | crt | + | C. acetobutylicum |
| butyryl-CoA | bcd | + | C. acetobutylicum, |
| dehydrogenase | | | M. elsdenii |
| electron transfer | etfAB | + | C. acetobutylicum, |
| flavoprotein | | | M. elsdenii |
| aldehyde/alcohol | adhE2 | + | C. acetobutylicum |
| dehydrogenase | | | |
| crotonyl-coA | ccr | + | S. coelicolor |
| reductase | | | |

* knockout or a reduction in expression are optional in the synthesis of the product, however, such knockouts increase various substrate intermediates and improve yield.

Exemplary Yield Data for E. coli Comprising Overexpression of atoB (EC), hbd (CA), crt (CA), bcd (CA), etfAB (CA), and adhE2 (CA)

| Knockout | | | | | Butanol | | Glucose | | Yield |
|---|---|---|---|---|---|---|---|---|---|
| adh | ldh | frd | fnr | pta | (mM) | (mg/L) | (mM) | (mg/L) | (g/g) |
| | | | | | 1.9 | 140.8 | 44.9 | 8089.2 | 0.02 |
| Δ | Δ | Δ | | | 3.7 | 274.2 | 30.7 | 5530.9 | 0.05 |
| Δ | Δ | Δ | Δ | | 2.1 | 155.7 | 22.2 | 3999.6 | 0.04 |
| Δ | Δ | Δ | | Δ | 2.7 | 200.1 | 28.2 | 5080.5 | 0.04 |
| Δ | Δ | Δ | Δ | Δ | 5 | 370.6 | 42.8 | 7710.8 | 0.05 |

Media: M9 + 2% glucose + 0.1% casamino acid + 0.1M MOPS + Trace metal mix + 0.1 mM IPTG, 37° C., 24 hr. (CA = C. acetobutylicum; EC = E. coli)

The disclosure provides recombinant microorganism comprising a biosynthetic pathway that provides a yield of greater than 0.015 grams of n-butanol per gram of glucose. For example, the recombinant microorganism can produce about 0.015 to about 0.060 grams of n-butanol per gram of glucose (e.g., greater than about 0.050, about 0.020 to about 0.050, about 0.030 to 0.040, and any ranges or values therebetween). In one embodiment, the parental microorganism does not produced n-butanol. In yet another embodiment, the parental microorganism produced only trace amounts of n-butanol (e.g., less than 0.010 grams of n-butanol per gram of glucose). In a specific embodiment the microorganism is an E. coli. In another aspect, the a culture comprises a population microorganism that is substantially homogenous (e.g., from about 70-100% homogenous). In another aspect, a culture can comprises a combination of micoorganism each having distinct biosynthetic pathways that produced metabolites that can be used by at least on other microorganism in culture in the production of n-butanol.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Ethanol Dehydrogenase (also referred to as Aldehyde-alcohol dehydrogenase) is encoded in E. coli by adhE. adhE comprises three activities: alcohol dehydrogenase (ADH); acetaldehyde/acetyl-CoA dehydrogenase (ACDH); pyruvate-formate-lyase deactivase (PFL deactivase); PFL deactivase activity catalyzes the quenching of the pyruvate-formate-lyase catalyst in an iron, NAD, and CoA dependent reaction. Homologs are known in the art (see, e.g., aldehyde-alcohol dehydrogenase (Polytomella sp. Pringsheim 198.80) gi|40644910|emb|CAD42653.2|(40644910); aldehyde-alcohol dehydrogenase (Clostridium botulinum A str. ATCC 3502) gi|148378348|ref|YP_{—}001252889.1|(148378348); aldehyde-alcohol dehydrogenase (Yersinia pestis C092) gi|16122410|ref|NP_{—}405723.1|(16122410); aldehyde-alcohol dehydrogenase (Yersinia pseudotuberculosis IP 32953) gi|51596429|ref|YP_{—}070620.1|(51596429); aldehyde-alcohol dehydrogenase (Yersinia pestis CO92) gi|115347889|emb|CAL20810.1|(115347889); aldehyde-alcohol dehydrogenase (Yersinia pseudotuberculosis IP 32953) gi|51589711|emb|CAH21341.1|(51589711); Aldehyde-alcohol dehydrogenase (Escherichia coli CFT073) gi|26107972|gb|AAN80172.1|AE016760_{—}31(26107972); aldehyde-alcohol dehydrogenase (Yersinia pestis biovar Microtus str. 91001) gi|45441777|ref|NP_{—}993316.1|(454-41777); aldehyde-alcohol dehydrogenase (Yersinia pestis biovar Microtus str. 91001) gi|45436639|gb|AAS62193.1|(45436639); aldehyde-alcohol dehydrogenase (Clostridium perfringens ATCC 13124) gi|110798574|ref|YP_{—}697219.1|(110798574); aldehyde-alcohol dehydrogenase (Shewanella oneidensis MR-1)gi|24373696|ref|NP_{—}717739.1|(24373696); aldehyde-alcohol dehydrogenase (Clostridium botulinum A str. ATCC 19397) gi|153932445|ref|YP_{—}001382747.1|(153932445); aldehyde-alcohol dehydrogenase (Yersinia pestis biovar Antiqua str. E1979001) gi|165991833|gb|EDR44134.1|(165991833); aldehyde-alcohol dehydrogenase (Clostridium botulinum A str. Hall) gi|153937530|ref|YP_{—}001386298.1|(153937530); aldehyde-alcohol dehydrogenase (Clostridium perfringens ATCC 13124) gi|110673221|gb|ABG82208.1|(110673221); aldehyde-alcohol dehydrogenase (Clostridium botulinum A str. Hall) gi|152933444|gb|ABS38943.1|(152933444); aldehyde-alcohol dehydrogenase (Yersinia pestis biovar Orientalis str. F1991016) gi|165920640|gb|EDR37888.1|(165920640); aldehyde-alcohol dehydrogenase (Yersinia pestis biovar Orientalis str. IP275)gi|165913933|gb|EDR32551.1|(165913933); aldehyde-alcohol dehydrogenase (Yersinia pestis Angola) gi|162419116|ref|YP_{—}001606617.1|(162419116); aldehyde-alcohol dehydrogenase (Clostridium botulinum F str. Langeland) gi|153940830|ref|YP_{—}001389712.1|(153940830); aldehyde-alcohol dehydrogenase (Escherichia coli HS) gi|157160746|ref|YP_{—}001458064.1|(157160746); aldehyde-alcohol dehydrogenase (Escherichia coli E24377A) gi|157155679|ref|YP_{—}001462491.1|(157155679); aldehyde-alcohol dehydrogenase (Yersinia enterocolitica subsp. enterocolitica 8081) gi|123442494|ref|YP_{—}001006472.1|(123442494); aldehyde-alcohol dehydrogenase (Synechococcus sp. JA-3-3Ab) gi|86605191|ref|YP_{—}473954.1|(86605191); aldehyde-alcohol dehydrogenase (Listeria monocytogenes str. 4b F2365) gi|46907864|ref|YP_{—}014253.1|(46907864); aldehyde-alcohol dehydrogenase (Enterococcus faecalis V583) gi|29375484|ref|NP_{—}814638.1|(29375484); aldehyde-alcohol dehydrogenase (Streptococcus agalactiae 2603V/R) gi|22536238|ref|NP_{—}687089.1|(22536238); aldehyde-alcohol dehydrogenase (Clostridium botulinum A str. ATCC 19397) gi|152928489|gb|ABS33989.1|(152928489); aldehyde-alcohol dehydrogenase (Escherichia coli E24377A) gi|157077709|gb|ABV17417.1|(157077709); aldehyde-alcohol dehydrogenase (Escherichia coli HS) gi|157066426|gb|ABV05681.1|(157066426); aldehyde-alcohol dehydrogenase (Clostridium botulinum F str. Langeland) gi|152936726|gb|ABS42224.1|(152936726); aldehyde-alcohol dehydrogenase (Yersinia pestis CA88-4125) gi|149292312|gb|EDM42386.1|(149292312); aldehyde-alcohol dehydrogenase (Yersinia enterocolitica subsp. enterocolitica 8081) gi|122089455|emb|CAL12303.1|(122089455); aldehyde-alcohol dehydrogenase (Chlamydomonas reinhardtii) gi|92084840|emb|CAF04128.1|(92084840); aldehyde-alcohol dehydrogenase (Synechococcus sp. JA-3-3Ab) gi|86553733|gb|ABC98691.1|(86553733); aldehyde-alcohol dehydrogenase (Shewanella oneidensis MR-1) gi|24348056|gb|AAN55183.1|AE015655_{—}9(24348056); aldehyde-alcohol dehydrogenase (Enterococcus faecalis V583) gi|29342944|gb|AA080708.1|(29342944); aldehyde-alcohol dehydrogenase (Listeria monocytogenes str. 4b F2365) gi|46881133|gb|AAT04430.1|(46881133); aldehyde-alcohol dehydrogenase (Listeria monocytogenes str. 1/2a F6854) gi|47097587|ref|ZP_{—}00235115.1|(47097587); aldehyde-alcohol dehydrogenase (Listeria monocytogenes str. 4b H7858) gi|47094265|ref|ZP_{—}00231973.1|(47094265); aldehyde-alcohol dehydrogenase (Listeria monocytogenes str. 4b H7858) gi|47017355|gblEAL08180.1|(47017355); aldehyde-alcohol dehydrogenase (Listeria monocytogenes str. 1/2a F6854) gi|47014034|gb|EAL05039.1|(47014034); aldehyde-alcohol dehydrogenase (Streptococcus agalactiae 2603V/R) gi|22533058|gb|AAM98961.1|AE014194_{—}6(22533058)ₚ; aldehyde-alcohol dehydrogenase (Yersinia pestis biovar Antiqua str. E1979001) gi|166009278|ref|ZP_{—}02230176.1|(166009278); aldehyde-alcohol dehydrogenase (Yersinia pestis biovar Orientalis str. IP275) gi|165938272|ref|ZP_{—}02226831.1|(165938272); aldehyde-alcohol dehydrogenase (Yersinia pestis biovar Orientalis str. F1991016) gi|165927374|ref|ZP_{—}02223206.1|(165927374); aldehyde-alcohol dehydrogenase (Yersinia pestis Angola) gi|162351931|gb|ABX85879.1|(162351931); aldehyde-alcohol dehydrogenase (Yersinia pseudotuberculosis IP 31758) gi|153949366|ref|YP_{—}001400938.1|(153949366); aldehyde-alcohol dehydrogenase (Yersinia pseudotuberculosis IP 31758) gi|152960861|gb|ABS48322.1|(152960861); aldehyde-alcohol dehydrogenase (Yersinia pestis CA88-4125) gi|149365899|ref|ZP_{—}01887934.1|(149365899); Acetaldehyde dehydrogenase (acetylating) (Escherichia coli CFT073) gi|26247570|ref|NP_{—}753610.1|(26247570); aldehyde-alcohol dehydrogenase (includes: alcohol dehydrogenase; acetaldehyde dehydrogenase (acetylating) (EC 1.2.1.10) (acdh); pyruvate-formate-lyase deactivase (pfl deactivase)) (Clostridium botulinum A str. ATCC 3502) gi|148287832|emb|CAL81898.1|(148287832); aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152980|sp|P0A9Q7.2|ADHE_ECOLI(71152980); aldehyde-alcohol dehydrogenase (includes: alcohol dehydrogenase and acetaldehyde dehydrogenase, and pyruvate-formate-lyase deactivase (Erwinia carotovora subsp. atroseptica SCR11043) gi|50121254|ref|YP_{—}050421.1|(50121254); aldehyde-alcohol dehydrogenase (includes: alcohol dehydrogenase and acetaldehyde dehydrogenase, and pyruvate-formate-lyase deactivase (Erwinia carotovora subsp. atroseptica SCR11043) gi|49611780|emb|CAG75229.1|(49611780); Aldehyde-alcohol dehydrogenase (Includes: Alcohol dehydrogenase (ADH); Acetaldehyde dehydrogenase (acetylating) (ACDH)) gi|19858620|sp|P33744.3|ADHE_CLOAB(19858620); Aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152683|sp|P0A9Q8.2|ADHE_ECO57(71152683); aldehyde-alcohol dehydrogenase (includes: alcohol dehydrogenase; acetaldehyde dehydrogenase (acetylating); pyruvate-formate-lyase deactivase (Clostridium difficile 630) gi|126697906|ref|YP_{—}001086803.1|(126697906); aldehyde-alcohol dehydrogenase (includes: alcohol dehydrogenase; acetaldehyde dehydrogenase (acetylating); pyruvate-formate-lyase deactivase (Clostridium difficile 630) gi|115249343|emb|CAJ67156.1|(115249343); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TTO1) gi|37526388|ref|NP_{—}929732.1|(37526388); aldehyde-alcohol dehydrogenase 2 (includes: alcohol dehydrogenase; acetaldehyde dehydrogenase) (Streptococcus pyogenes str. Manfredo) gi|134271169|emb|CAM29381.1|(134271169); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TTO1) gi|36785819|emb|CAE14870.1|(36785819); aldehyde-alcohol dehydrogenase (includes: alcohol dehydrogenase and pyruvate-formate-lyase deactivase (Clostridium difficile 630) gi|126700586|ref|YP_{—}001089483.1|(126700586); aldehyde-alcohol dehydrogenase (includes: alcohol dehydrogenase and pyruvate-formate-lyase deactivase (Clostridium difficile 630) gi|115252023|emb|CAJ69859.1|(115252023); aldehyde-alcohol dehydrogenase 2 (Streptococcus pyogenes str. Manfredo) gi|139472923|ref|YP_{—}001127638.1|(139472923); aldehyde-alcohol dehydrogenase E (Clostridium perfringens str. 13) gi|18311513|ref|NP-563447.1|(18311513); aldehyde-alcohol dehydrogenase E (Clostridium perfringens str. 13) gi|18146197|dbj|BAB82237.1|(18146197); Aldehyde-alcohol dehydrogenase, ADHE1 (Clostridium acetobutylicum ATCC 824) gi|15004739|ref|NP_{—}149199.1|(15004739); Aldehyde-alcohol dehydrogenase, ADHE1 (Clostridium acetobutylicum ATCC 824) gi|14994351|gb|AAK76781.1|AE001438_{—}34(14994351); Aldehyde-alcohol dehydrogenase 2 (Includes: Alcohol dehydrogenase (ADH); acetaldehyde/acetyl-CoA dehydrogenase (ACDH)) gi|2492737|sp|Q24803.1|ADH2_ENTHI(2492737); alcohol dehydrogenase (Salmonella enterica subsp. enterica serovar Typhi str. CT18) gi|16760134|ref|NP_{—}455751.1|(16760134); and alcohol dehydrogenase (Salmonella enterica subsp. enterica serovar Typhi) gi|16502428|emb|CAD08384.1|(16502428)), each sequence associated with the accession number is incorporated herein by reference in its entirety.

Lactate Dehydrogenase (also referred to as D-lactate dehydrogenase and fermentive dehydrognase) is encoded in E. coli by ldhA and catalyzes the NADH-dependent conversion of pyruvate to D-lactate. ldhA homologs and variants are known. In fact there are currently 1664 bacterial lactate dehydrogenases available through NCBI. For example, such homologs and variants include, for example, D-lactate dehydrogenase (D-LDH) (Fermentative lactate dehydrogenase) gi|1730102|sp|P52643.1|LDHD_ECOLI(1730102); D-lactate dehydrogenase gi|1049265|gb|AAB51772.1|(1049265); D-lactate dehydrogenase (Escherichia coli APEC O1) gi|117623655|ref|YP_{—}852568.1|(117623655); D-lactate dehydrogenase (Escherichia coli CFT073) gi|26247689|ref|NP_{—}753729.1|(26247689); D-lactate dehydrogenase (Escherichia coli O157:H7 EDL933) gi|15801748|ref|NP_{—}287766.1|(15801748); D-lactate dehydrogenase (Escherichia coli APEC 01) gi|115512779|gb|ABJ00854.1|(115512779); D-lactate dehydrogenase (Escherichia coli CFT073) gi|26108091|gb|AAN80291.1|AE016760_{—}150(26108091); fermentative D-lactate dehydrogenase, NAD-dependent (Escherichia coli K12) gi|16129341|ref|NP_{—}415898.1|(16129341); fermentative D-lactate dehydrogenase, NAD-dependent (Escherichia coli UTI89) gi|91210646|ref|YP_{—}540632.1|(91210646); fermentative D-lactate dehydrogenase, NAD-dependent (Escherichia coli K12) gi|1787645|gb|AAC74462.1|(1787645); fermentative D-lactate dehydrogenase, NAD-dependent (Escherichia coli W3110) gi|89108227|ref|AP_{—}002007.1|(89108227); fermentative D-lactate dehydrogenase, NAD-dependent (Escherichia coli W3110) gi|1742259|dbj|BAA14990.1|(1742259); fermentative D-lactate dehydrogenase, NAD-dependent (Escherichia coli UTI89) gi|91072220|gb|ABE07101.1|(91072220); fermentative D-lactate dehydrogenase, NAD-dependent (Escherichia coli O157:H7 EDL933) gi|12515320|gb|AAG56380.1|AE005366_{—}6(12515320); fermentative D-lactate dehydrogenase (Escherichia coli O157:H7 str. Sakai) gi|13361468|dbj|BAB35425.1|(13361468); COG1052: Lactate dehydrogenase and related dehydrogenases (Escherichia coli 101-1) gi|83588593|ref|ZP_{—}00927217.1|(83588593); COG1052: Lactate dehydrogenase and related dehydrogenases (Escherichia coli 53638) gi|75515985|ref|ZP_{—}00738103.1|(75515985); COG1052: Lactate dehydrogenase and related dehydrogenases (Escherichia coli E22) gi|75260157|ref|ZP_{—}00731425.1|(75260157); COG1052: Lactate dehydrogenase and related dehydrogenases (Escherichia coli F11) gi|75242656|ref|ZP_{—}00726400.1|(75242656); COG1052: Lactate dehydrogenase and related dehydrogenases (Escherichia coli E110019) gi|75237491|ref|ZP_{—}00721524.1|(75237491); COG1052: Lactate dehydrogenase and related dehydrogenases (Escherichia coli B7A) gi|75231601|ref|ZP_{—}00717959.1|(75231601); and COG1052: Lactate dehydrogenase and related dehydrogenases (Escherichia coli B171) gi|75211308|ref|ZP_{—}00711407.1|(75211308), each sequence associated with the accession number is incorporated herein by reference in its entirety.

Two membrane-bound, FAD-containing enzymes are responsible for the catalysis of fumarate and succinate interconversion; the fumarate reductase is used in anaerobic growth, and the succinate dehydrogenase is used in aerobic growth. Fumarate reductase comprises multiple subunits (e.g., frdA, B, and C in E. coli). Modification of any one of the subunits can result in the desired activity herein. For example, a knockout of frdB, frdC or frdBC is useful in the methods of the disclosure. Frd homologs and variants are known. For example, homologs and variants includes, for example, Fumarate reductase subunit D (Fumarate reductase 13 kDa hydrophobic protein) gi|67463543|sp|P0A8Q3.1|FRDD_ECOLI(67463543); Fumarate reductase subunit C (Fumarate reductase 15 kDa hydrophobic protein) gi|1346037|sp|P20923.2|FRDC_PROVU(1346037); Fumarate reductase subunit D (Fumarate reductase 13 kDa hydrophobic protein) gi|120499|sp|P20924.1|FRDD_PROVU(120499); Fumarate reductase subunit C (Fumarate reductase 15 kDa hydrophobic protein) gi|67463538|sp|POA8Q0.1|FRDC_ECOLI(67463538); fumarate reductase iron-sulfur subunit (Escherichia coli) gi|145264|gb|AAA23438.1|(145264); fumarate reductase flavoprotein subunit (Escherichia coli) gi|145263|gb|AAA23437.1|(145263); Fumarate reductase flavoprotein subunit gi|37538290|sp|P17412.3|FRDA_WOLSU(37538290); Fumarate reductase flavoprotein subunit gi|120489|sp|P00363.3|FRDA_ECOLI(120489); Fumarate reductase flavoprotein subunit gi|120490|sp|P20922.1|FRDA_PROVU(120490); Fumarate reductase flavoprotein subunit precursor (Flavocytochrome c) (Flavocytochrome c3) (Fcc3) gi|119370087|sp|Q07WU7.2|FRDA_SHEFN(119370087); Fumarate reductase iron-sulfur subunit gi|81175308|sp|POAC47.2|FRDB_ECOLI(81175308); Fumarate reductase flavoprotein subunit (Flavocytochrome c) (Flavocytochrome c3) (Fcc3) gi|119370088|sp|POC278.1|FRDA_SHEFR(119370088); Frd operon uncharacterized protein C gi|140663|sp|P20927.1|YFRC_PROVU(140663); Frd operon probable iron-sulfur subunit A gi|140661|sp|P20925.1|YFRA_PROVU(140661); Fumarate reductase iron-sulfur subunit gi|120493|sp|P20921.2|FRDB_PROVU(120493); Fumarate reductase flavoprotein subunit gi|2494617|sp|006913.2|FRDA_HELPY(2494617); Fumarate reductase flavoprotein subunit precursor (Iron(III)-induced flavocytochrome C3) (Ifc3) gi|13878499|sp|Q9Z4P0.1|FRD2_SHEFN(13878499); Fumarate reductase flavoprotein subunit gi|54041009|sp|P64174.1|FRDA_MYCTU(54041009); Fumarate reductase flavoprotein subunit gi|54037132|sp|P64175.1|FRDA_MYCBO(54037132); Fumarate reductase flavoprotein subunit gi|12230114|sp|Q9ZMP0.1|FRDA_HELPJ(12230114); Fumarate reductase flavoprotein subunit gi|1169737|sp|P44894.1|FRDA_HAEIN(1169737); fumarate reductase flavoprotein subunit (Wolinella succinogenes) gi|13160058|emb|CAA04214.2|(13160058); Fumarate reductase flavoprotein subunit precursor (Flavocytochrome c) (FL cyt) gi|25452947|sp|P83223.2|FRDA_SHEON(25452947); fumarate reductase iron-sulfur subunit (Wolinella succinogenes) gi|2282000|emb|CAA04215.1|(2282000); and fumarate reductase cytochrome b subunit (Wolinella succinogenes) gi|2281998|emb|CAA04213.1|(2281998), each sequence associated with the accession number is incorporated herein by reference in its entirety.

Phosphate acetyltransferase is encoded in E. coli by pta. PTA is involved in conversion of acetate to acetyl-CoA. Specifically, PTA catalyzes the conversion of acetyl-coA to acetyl-phosphate. PTA homologs and variants are known. There are approximately 1075 bacterial phosphate acetyltransferases available on NCBI. For example, such homologs and variants include phosphate acetyltransferase Pta (Rickettsia felis URRWXCal2) gi|67004021|gb|AAY60947.1|(67004021); phosphate acetyltransferase (Buchnera aphidicola str. Cc (Cinara cedri)) gi|116256910|gb|ABJ90592.1|(116256910); pta (Buchnera aphidicola str. Cc (Cinara cedri)) gi|116515056|ref|YP_{—}802685.1|(116515056); pta (Wigglesworthia glossinidia endosymbiont of Glossina brevipalpis) gi|25166135|dbj|BAC24326.1|(25166135); Pta (Pasteurella multocida subsp. multocida str. Pm70) gi|12720993|gb|AAK02789.1|(12720993); Pta (Rhodospirillum rubrum) gi|25989720|gb|AAN75024.1|(25989720); pta (Listeria welshimeri serovar 6b str. SLCC5334) gi|116742418|emb|CAK21542.1|(116742418); Pta (Mycobacterium avium subsp. paratuberculosis K-10) gi|41398816|gb|AAS06435.1|(41398816); phosphate acetyltransferase (pta) (Borrelia burgdorferi B31) gi|15594934|ref|NP_{—}212723.1|(15594934); phosphate acetyltransferase (pta) (Borrelia burgdorferi B31) gi|2688508|gb|AAB91518.1|(2688508); phosphate acetyltransferase (pta) (Haemophilus influenzae Rd KW20) gi|1574131|gb|AAC22857.1|(1574131); Phosphate acetyltransferase Pta (Rickettsia bellii RML369-C) gi|91206026|ref|YP_{—}538381.1|(91206026); Phosphate acetyltransferase Pta (Rickettsia bellii RML369-C) gi|91206025|ref|YP_{—}538380.1|(91206025); phosphate acetyltransferase pta (Mycobacterium tuberculosis F11) gi|148720131|gb|ABR04756.1|(148720131); phosphate acetyltransferase pta (Mycobacterium tuberculosis str. Haarlem) gi|134148886|gb|EBA40931.1|(134148886); phosphate acetyltransferase pta (Mycobacterium tuberculosis C) gi|124599819|gb|EAY58829.1|(124599819); Phosphate acetyltransferase Pta (Rickettsia bellii RML369-C) gi|91069570|gb|ABE05292.1|(91069570); Phosphate acetyltransferase Pta (Rickettsia bellii RML369-C) gi|91069569|gb|ABE05291.1|(91069569); phosphate acetyltransferase (pta) (Treponema pallidum subsp. pallidum str. Nichols) gi|15639088|ref|NP_{—}218534.1|(15639088); and phosphate acetyltransferase (pta) (Treponema pallidum subsp. pallidum str. Nichols) gi|3322356|gb|AAC65090.1|(3322356), each sequence associated with the accession number is incorporated herein by reference in its entirety.

Pyruvate-formate lyase (Formate acetylytransferase) is an enzyme that catalyzes the conversion of pyruvate to acetyl)-coA and formate. It is induced by pfl-activating enzyme under anaerobic conditions by generation of an organic free radical and decreases significantly during phosphate limitation. Formate acetylytransferase is encoded in E. coli by pflB. PFLB homologs and variants are known. For examples, such homologs and variants include, for example, Formate acetyltransferase 1 (Pyruvate formate-lyase 1) gi|129879|sp|P09373.2|PFLB_ECOLI(129879); formate acetyltransferase 1 (Yersinia pestis C092) gi|16121663|ref|NP_{—}404976.1|(16121663); formate acetyltransferase 1 (Yersinia pseudotuberculosis IP 32953) gi|51595748|ref|YP_{—}069939.1|(51595748); formate acetyltransferase 1 (Yersinia pestis biovar Microtus str. 91001) gi|454-41037|ref|NP_{—}992576.1|(454-41037); formate acetyltransferase 1 (Yersinia pestis C092) gi|115347142|emb|CAL20035.1|(115347142); formate acetyltransferase 1 (Yersinia pestis biovar Microtus str. 91001) gi|45435896|gb|AAS61453.1|(45435896); formate acetyltransferase 1 (Yersinia pseudotuberculosis IP 32953) gi|51589030|emb|CAH20648.1|(51589030); formate acetyltransferase 1 (Salmonella enterica subsp. enterica serovar Typhi str. CT18) gi|16759843|ref|NP_{—}455-460.1|(16759843); formate acetyltransferase 1 (Salmonella enterica subsp. enterica serovar Paratyphi A str. ATCC 9150) gi|56413977|ref|YP_{—}151052.1|(56413977); formate acetyltransferase 1 (Salmonella enterica subsp. enterica serovar Typhi) gi|16502136|emb|CAD05373.1|(16502136); formate acetyltransferase 1 (Salmonella enterica subsp. enterica serovar Paratyphi A str. ATCC 9150) gi|56128234|gb|AAV77740.1|(56128234); formate acetyltransferase 1 (Shigella dysenteriae Sd197) gi|82777577|ref|YP_{—}403926.1|(82777577); formate acetyltransferase 1 (Shigella flexneri 2a str. 2457T) gi|30062438|ref|NP_{—}836609.1|(30062438); formate acetyltransferase 1 (Shigella flexneri 2a str. 2457T) gi|30040684|gb|AAP16415.1|(30040684); formate acetyltransferase 1 (Shigella flexneri 5 str. 8401) gi|110614459|gb|ABF03126.1|(110614459); formate acetyltransferase 1 (Shigella dysenteriae Sd197) gi|81241725|gb|ABB62435.1|(81241725); formate acetyltransferase 1 (Escherichia coli O157:H7 EDL933) gi|12514066|gb|AAG55388.1|AE005279_{—}8(12514066); formate acetyltransferase 1 (Yersinia pestis KIM) gi|22126668|ref|NP_{—}670091.1|(22126668); formate acetyltransferase 1 (Streptococcus agalactiae A909) gi|76787667|ref|YP_{—}330335.1|(76787667); formate acetyltransferase 1 (Yersinia pestis KIM) gi|21959683|gb|AAM86342.1|AE013882_{—}3(21959683); formate acetyltransferase 1 (Streptococcus agalactiae A909) gi|76562724|gb|ABA45308.1|(76562724); formate acetyltransferase 1 (Yersinia enterocolitica subsp. enterocolitica 8081) gi|123441844|ref|YP_{—}001005827.1|(123441844); formate acetyltransferase 1 (Shigella flexneri 5 str. 8401) gi|110804911|ref|YP_{—}688431.1|(110804911); formate acetyltransferase 1 (Escherichia coli UTI89) gi|91210004|ref|YP_{—}539990.1|(91210004); formate acetyltransferase 1 (Shigella boydii Sb227) gi|82544641|ref|YP_{—}408588.1|(82544641); formate acetyltransferase 1 (Shigella sonnei Ss046) gi|74311459|ref|YP_{—}309878.1|(74311459); formate acetyltransferase 1 (Klebsiella pneumoniae subsp. pneumoniae MGH 78578) gi|152969488|ref|YP_{—}001334597.1|(152969488); formate acetyltransferase 1 (Salmonella enterica subsp. enterica serovar Typhi Ty2) gi|29142384|ref|NP_{—}805726.1|(29142384) formate acetyltransferase 1 (Shigella flexneri 2a str. 301) gi|24112311|ref|NP_{—}706821.1|(24112311); formate acetyltransferase 1 (Escherichia coli O157:H7 EDL933) gi|15800764|ref|NP_{—}286778.1|(15800764); formate acetyltransferase 1 (Klebsiella pneumoniae subsp. pneumoniae MGH 78578) gi|150954337|gb|ABR76367.1|(150954337); formate acetyltransferase 1 (Yersinia pestis CA88-4125) gi|149366640|ref|ZP_{—}01888674.1|(149366640); formate acetyltransferase 1 (Yersinia pestis CA88-4125) gi|149291014|gb|EDM41089.1|(149291014); formate acetyltransferase 1 (Yersinia enterocolitica subsp. enterocolitica 8081) gi|122088805|emb|CAL11611.1|(122088805); formate acetyltransferase 1 (Shigella sonnei Ss046) gi|73854936|gb|AAZ87643.1|(73854936); formate acetyltransferase 1 (Escherichia coli UTI89) gi|91071578|gb|ABE06459.1|(91071578); formate acetyltransferase 1 (Salmonella enterica subsp. enterica serovar Typhi Ty2) gi|29138014|gb|AA069575.1|(29138014); formate acetyltransferase 1 (Shigella boydii Sb227) gi|81246052|gb|ABB66760.1|(81246052); formate acetyltransferase 1 (Shigella flexneri 2a str. 301) gi|24051169|gb|AAN42528.1|(24051169); formate acetyltransferase 1 (Escherichia coli O157:H7 str. Sakai) gi|13360445|dbj|BAB34409.1|(13360445); formate acetyltransferase 1 (Escherichia coli O157:H7 str. Sakai) gi|15830240|ref|NP_{—}309013.1|(15830240); formate acetyltransferase I (pyruvate formate-lyase 1) (Photorhabdus luminescens subsp. laumondii TTO1) gi|36784986|emb|CAE13906.1|(36784986); formate acetyltransferase I (pyruvate formate-lyase 1) (Photorhabdus luminescens subsp. laumondii TTO1) gi|37525558|ref|NP_{—}928902.1|(37525558); formate acetyltransferase (Staphylococcus aureus subsp. aureus Mu50) gi|14245993|dbj|BAB56388.1|(14245993); formate acetyltransferase (Staphylococcus aureus subsp. aureus Mu50) gi|15923216|ref|NP_{—}370750.1|(15923216); Formate acetyltransferase (Pyruvate Formate-Lyase) gi|81706366|sp|Q7A7X6.1|PFLB_STAAN(81706366); Formate acetyltransferase (Pyruvate formate-lyase) gi|81782287|sp|Q99WZ7.1|PFLB_STAAM(81782287); Formate acetyltransferase (Pyruvate formate-lyase) gi|81704726|sp|Q7A1W9.1|PFLB_STAAW(81704726); formate acetyltransferase (Staphylococcus aureus subsp. aureus Mu3) gi|156720691|dbj|BAF77108.1|(156720691); formate acetyltransferase (Erwinia carotovora subsp. atroseptica SCR11043) gi|50121521|ref|YP_{—}050688.1|(50121521); formate acetyltransferase (Erwinia carotovora subsp. atroseptica SCR11043) gi|49612047|emb|CAG75496.1|(49612047); formate acetyltransferase (Staphylococcus aureus subsp. aureus str. Newman) gi|150373174|dbj↑BAF66434.1|(150373174); formate acetyltransferase (Shewanella oneidensis MR-1) gi|24374439|ref|NP_{—}718482.1|(24374439); formate acetyltransferase (Shewanella oneidensis MR-1) gi|24349015|gb|AAN55926.1|AE015730_{—}3(24349015); formate acetyltransferase (Actinobacillus pleuropneumoniae serovar 3 str. JL03) gi|165976461|ref|YP_{—}001652054.1|(165976461); formate acetyltransferase (Actinobacillus pleuropneumoniae serovar 3 str. JL03) gi|165876562|gb|ABY69610.1|(165876562); formate acetyltransferase (Staphylococcus aureus subsp. aureus MW2) gi|21203365|dbj|BAB94066.1|(21203365); formate acetyltransferase (Staphylococcus aureus subsp. aureus N315) gi|13700141|dbj|BAB41440.1|(13700141); formate acetyltransferase (Staphylococcus aureus subsp. aureus str. Newman) gi|151220374|ref|YP_{—}001331197.1|(151220374); formate acetyltransferase (Staphylococcus aureus subsp. aureus Mu3) gi|156978556|ref|YP_{—}001440815.1|(156978556); formate acetyltransferase (Synechococcus sp. JA-2-3B′a(2-13)) gi|86607744|ref|YP_{—}476506.1|(86607744); formate acetyltransferase (Synechococcus sp. JA-3-3Ab) gi|86605195|ref|YP_{—}473958.1|(86605195); formate acetyltransferase (Streptococcus pneumoniae D39) gi|116517188|ref|YP_{—}815928.1|(116517188); formate acetyltransferase (Synechococcus sp. JA-2-3B′a(2-13)) gi|86556286|gb|ABD01243.1|(86556286); formate acetyltransferase (Synechococcus sp. JA-3-3Ab) gi|86553737|gb|ABC98695.1|(86553737); formate acetyltransferase (Clostridium novyi NT) gi|118134908|gb|ABK61952.1|(118134908); formate acetyltransferase (Staphylococcus aureus subsp. aureus MRSA252) gi|49482458|ref|YP_{—}039682.1|(49482458); and formate acetyltransferase (Staphylococcus aureus subsp. aureus MRSA252) gi|49240587|emb|CAG39244.1|(49240587), each sequence associated with the accession number is incorporated herein by reference in its entirety.

FNR transcriptional dual regulators are transcription requlators responsive to oxygen content. FNR is an anaerobic regulator that represses the expression of PDHc. Accordingly, reducing FNR will result in an increase in PDHc expression. FNR homologs and variants are known. For examples, such homologs and variants include, for example, DNA-binding transcriptional dual regulator, global regulator of anaerobic growth (Escherichia coli W3110) gi|1742191|dbj|BAA14927.1|(1742191); DNA-binding transcriptional dual regulator, global regulator of anaerobic growth (Escherichia coli K12) gi|16129295|ref|NP_{—}415850.1|(16129295); DNA-binding transcriptional dual regulator, global regulator of anaerobic growth (Escherichia coli K12) gi|1787595|gb|AAC74416.1|(1787595); DNA-binding transcriptional dual regulator, global regulator of anaerobic growth (Escherichia coli W3110) gi|89108182|ref|AP_{—}001962.1|(89108182); fumarate/nitrate reduction transcriptional regulator (Escherichia coli UTI89) gi|162138444|ref|YP_{—}540614.2|(162138444); fumarate/nitrate reduction transcriptional regulator (Escherichia coli CFT073) gi|161486234|ref|NP_{—}753709.2|(161486234); fumarate/nitrate reduction transcriptional regulator (Escherichia coli O157:H7 EDL933) gi|15801834|ref|NP_{—}287852.1|(15801834); fumarate/nitrate reduction transcriptional regulator (Escherichia coli APEC O1) gi|117623587|ref|YP_{—}852500.1|(117623587); fumarate and nitrate reduction regulatory protein gi|71159334|sp|P0A9E5.1|FNR_ECOLI(71159334); transcriptional regulation of aerobic, anaerobic respiration, osmotic balance (Escherichia coli O157:H7 EDL933) gi|12515424|gb|AAG56466.1|AE005372_{—}11(12515424); Fumarate and nitrate reduction regulatory protein gi|71159333|sp|P0A9E6.1|FNR_ECOL6(71159333); Fumarate and nitrate reduction Regulatory protein (Escherichia coli CFT073) gi|26108071|gb|AAN80271.1|AE016760_{—}130(26108071); fumarate and nitrate reduction regulatory protein (Escherichia coli UTI89) gi|91072202|gb|ABE07083.1|(91072202); fumarate and nitrate reduction regulatory protein (Escherichia coli HS) gi|157160845|ref|YP_{—}001458163.1|(157160845); fumarate and nitrate reduction regulatory protein (Escherichia coli E24377A) gi|157157974|ref|YP_{—}001462642.1|(157157974); fumarate and nitrate reduction regulatory protein (Escherichia coli E24377A) gi|157080004|gb|ABV19712.1|(157080004); fumarate and nitrate reduction regulatory protein (Escherichia coli HS) gi|157066525|gb|ABV05780.1|(157066525); fumarate and nitrate reduction regulatory protein (Escherichia coli APEC O1) gi|115512711|gb|ABJ00786.1|(115512711); transcription regulator Fnr (Escherichia coli O157:H7 str. Sakai) gi|13361380|dbj|BAB35338.1|(13361380) DNA-binding transcriptional dual regulator (Escherichia coli K12) gi|16131236|ref|NP_{—}417816.1|(16131236), to name a few, each sequence associated with the accession number is incorporated herein by reference in its entirety.

An acetoacetyl-coA thiolase (also sometimes referred to as an acetyl-coA acetyltransferase) catalyzes the production of acetoacetyl-coA from two molecules of acetyl-coA. Depending upon the organism used a heterologous acetoacetyl-coA thiolase (acetyl-coA acetyltransferase) can be engineered for expression in the organism. Alternatively a native acetoacetyl-coA thiolase (acetyl-coA acetyltransferase) can be overexpressed. Acetoacetyl-coA thiolase is encoded in E. coli by thl. Acetyl-coA acetyltransferase is encoded in C. acetobutylicum by atoB. THL and AtoB homologs and variants are known. For examples, such homologs and variants include, for example, acetyl-coa acetyltransferase (thiolase) (Streptomyces coelicolor A3(2)) gi|21224359|ref|NP_{—}630138.1|(21224359); acetyl-coa acetyltransferase (thiolase) (Streptomyces coelicolor A3(2)) gi|3169041|emb|CAA19239.1|(3169041); Acetyl CoA acetyltransferase (thiolase) (Alcanivorax borkumensis SK2) gi|110834428|ref|YP_{—}693287.1|(110834428); Acetyl CoA acetyltransferase (thiolase) (Alcanivorax borkumensis SK2) gi|110647539|emb|CAL17015.1|(110647539); acetyl CoA acetyltransferase (thiolase) (Saccharopolyspora erythraea NRRL 2338) gi|133915420|emb|CAM05533.1|(133915420); acetyl-coa acetyltransferase (thiolase) (Saccharopolyspora erythraea NRRL 2338) gi|134098403|ref|YP_{—}001104064.1|(134098403); acetyl-coa acetyltransferase (thiolase) (Saccharopolyspora erythraea NRRL 2338) gi|133911026|emb|CAM01139.1|(133911026); acetyl-CoA acetyltransferase (thiolase) (Clostridium botulinum A str. ATCC 3502) gi|148290632|emb|CAL84761.1|(148290632); acetyl-CoA acetyltransferase (thiolase) (Pseudomonas aeruginosa UCBPP-PA14) gi|115586808|gb|ABJ12823.1|(115586808); acetyl-CoA acetyltransferase (thiolase) (Ralstonia metallidurans CH₃₄) gi|93358270|gb|ABF12358.1|(93358270); acetyl-CoA acetyltransferase (thiolase) (Ralstonia metallidurans CH₃₄) gi|93357190|gb|ABF11278.1|(93357190); acetyl-CoA acetyltransferase (thiolase) (Ralstonia metallidurans CH₃₄) gi|93356587|gb|ABF10675.1|(93356587); acetyl-CoA acetyltransferase (thiolase) (Ralstonia eutropha JMP134) gi|72121949|gb|AAZ64135.1|(72121949); acetyl-CoA acetyltransferase (thiolase) (Ralstonia eutropha JMP134)gi|72121729|gb|AAZ63915.1|(72121729); acetyl-CoA acetyltransferase (thiolase) (Ralstonia eutropha JMP134) gi|72121320|gb|AAZ63506.1|(72121320); acetyl-CoA acetyltransferase (thiolase) (Ralstonia eutropha JMP134) gi|72121001|gb|AAZ63187.1|(72121001); acetyl-CoA acetyltransferase (thiolase) (Escherichia coli) gi|2764832|emb|CAA66099.1|(2764832), each sequence associated with the accession number is incorporated herein by reference in its entirety.

3 hydroxy-butyryl-coA-dehydrogenase catalyzes the conversion of acetoacetyl-coA to 3-hydroxybutyryl-CoA. Depending upon the organism used a heterologous 3-hydroxy-butyryl-coA-dehydrogenase can be engineered for expression in the organism. Alternatively a native 3-hydroxy-butyryl-coA-dehydrogenase can be overexpressed. 3-hydroxy-butyryl-coA-dehydrogenase is encoded in C. acetobuylicum by hbd. HBD homologs and variants are known. For examples, such homologs and variants include, for example, 3-hydroxybutyryl-CoA dehydrogenase (Clostridium acetobutylicum ATCC 824) gi|15895965|ref|NP_{—}349314.1|(15895965); 3-hydroxybutyryl-CoA dehydrogenase (Bordetella pertussis Tohama I) gi|33571103|emb|CAE40597.1|(33571103); 3-hydroxybutyryl-CoA dehydrogenase (Streptomyces coelicolor A3(2)) gi|21223745|ref|NP_{—}629524.1|(21223745); 3-hydroxybutyryl-CoA dehydrogenase gi|1055222|gb|AAA95971.1|(1055222); 3-hydroxybutyryl-CoA dehydrogenase (Clostridium perfringens str. 13) gi|18311280|ref|NP_{—}563214.1|(18311280); 3-hydroxybutyryl-CoA dehydrogenase (Clostridium perfringens str. 13) gi|18145963|dbj|BAB82004.1|(18145963) each sequence associated with the accession number is incorporated herein by reference in its entirety.

Crotonase catalyzes the conversion of 3-hydroxybutyryl-CoA to crotonyl-CoA. Depending upon the organism used a heterologous Crotonase can be engineered for expression in the organism. Alternatively a native Crotonase can be overexpressed. Crotonase is encoded in C. acetobuylicum by crt. CRT homologs and variants are known. For examples, such homologs and variants include, for example, crotonase (butyrate-producing bacterium L2-50) gi|119370267|gb|ABL68062.1|(119370267); crotonase gi|1055218|gb|AAA95967.1|(1055218); crotonase (Clostridium perfringens NCTC 8239) gi|168218170|ref|ZP_{—}02643795.1|(168218170); crotonase (Clostridium perfringens CPE str. F4969) gi|168215036|ref|ZP_{—}02640661.1|(168215036); crotonase (Clostridium perfringens E str. JGS1987) gi|168207716|ref|ZP_{—}02633721.1|(168207716); crotonase (Azoarcus sp. EbN1) gi|56476648|ref|YP_{—}158237.1|(56476648); crotonase (Roseovarius sp. TM1035) gi|149203066|ref|ZP_{—}01880037.1|(149203066); crotonase (Roseovarius sp. TM1035) gi|1491-43612|gb|EDM31648.1|(149143612); crotonase; 3-hydroxbutyryl-CoA dehydratase (Mesorhizobium loti MAFF303099) gi|14027492|dbj|BAB53761.1|(14027492); crotonase (Roseobacter sp. SK209-2-6) gi|126738922|ref|ZP_{—}01754618.1|(126738922); crotonase (Roseobacter sp. SK209-2-6) gi|126720103|gb|EBA16810.1|(126720103); crotonase (Marinobacter sp. ELB17) gi|126665001|ref|ZP_{—}01735984.1|(126665001); crotonase (Marinobacter sp. ELB17) gi|126630371|gb|EBA00986.1|(126630371); crotonase (Azoarcus sp. EbN1) gi|56312691|emb|CAI07336.1|(56312691); crotonase (Marinomonas sp. MED121) gi|86166463|gb|EAQ67729.1|(86166463); crotonase (Marinomonas sp. MED121) gi|87118829|ref|ZP_{—}01074728.1|(87118829); crotonase (Roseovarius sp. 217) gi|85705898|ref|ZP_{—}01036994.1|(85705898); crotonase (Roseovarius sp. 217) gi|85669486|gb|EAQ24351.1|(85669486); crotonase gi|1055218|gb|AAA95967.1|(1055218); 3-hydroxybutyryl-CoA dehydratase (Crotonase) gi|1706153|sp|P52046.1|CRT_CLOAB(1706153); Crotonase (3-hydroxybutyryl-COA dehydratase) (Clostridium acetobutylicum ATCC 824) gi|15025745|gb|AAK80658.1|AE007768_{—}12(15025745) each sequence associated with the accession number is incorporated herein by reference in its entirety.

Butyryl-coA dehydrogenase is an enzyme in the protein pathway that catalyzes the reduction of crotonyl-CoA to butyryl-CoA. A butyryl-CoA dehydrogenase complex (Bcd/EtfAB) couples the reduction of crotonyl-CoA to butyryl-CoA with the reduction of ferredoxin. Depending upon the organism used a heterologous butyryl-CoA dehydrogenase can be engineered for expression in the organism. Alternatively, a native butyryl-CoA dehydrogenase can be overexpressed. Butyryl-coA dehydrognase is encoded in C. acetobuylicum and M. elsdenii by bcd. BCD homologs and variants are known. For examples, such homologs and variants include, for example, butyryl-CoA dehydrogenase (Clostridium acetobutylicum ATCC 824) gi|15895968|ref|NP_{—}349317.1|(15895968); Butyryl-CoA dehydrogenase (Clostridium acetobutylicum ATCC 824) gi|15025744|gb|AAK80657.1|AE007768_{—}11(15025744); butyryl-CoA dehydrogenase (Clostridium botulinum A str. ATCC 3502) gi|148381147|ref|YP_{—}001255688.1|(148381147); butyryl-CoA dehydrogenase (Clostridium botulinum A str. ATCC 3502) gi|148290631|emb|CAL84760.1|(148290631), each sequence associated with the accession number is incorporated herein by reference in its entirety. BCD can be expressed in combination with a flavoprotein electron transfer protein. Useful flavoprotein electron transfer protein subunits are expressed in C. acetobutylicum and M. elsdenii by a gene etfA and etfB (or the operon etfAB). ETFA, B, and AB homologs and variants are known. For examples, such homologs and variants include, for example, putative a-subunit of electron-transfer flavoprotein gi|1055221|gb|AAA95970.1|(1055221); putative b-subunit of electron-transfer flavoprotein gi|1055220|gb|AAA95969.1|(1055220), each sequence associated with the accession number is incorporated herein by reference in its entirety.

Aldehyde/alcohol dehydrogenase catalyzes the conversion of butyryl-CoA to butyraldehyde and butyraldehyde to 1-butanol. In one aspect, the aldehyde/alcohol dehydrogenase preferentially catalyzes the conversion of butyryl-CoA to butyraldehyde and butyraldehyde to 1-butanol. Depending upon the organism used a heterologous aldehyde/alcohol dehydrogenase can be engineered for expression in the organism. Alternatively, a native aldehyde/alcohol dehydrogenase can be overexpressed. aldehyde/alcohol dehydrogenase is encoded in C. acetobuylicum by adhE (e.g., an adhE2). ADHE (e.g., ADHE2) homologs and variants are known. For examples, such homologs and variants include, for example, aldehyde-alcohol dehydrogenase (Clostridium acetobutylicum) gi|3790107|gb|AAD04638.1|(3790107); aldehyde-alcohol dehydrogenase (Clostridium botulinum A str. ATCC 3502) gi|148378348|ref|YP_{—}001252889.1|(148378348); Aldehyde-alcohol dehydrogenase (Includes: Alcohol dehydrogenase (ADH) Acetaldehyde dehydrogenase (acetylating) (ACDH) gi|19858620|sp|P33744.3|ADHE_CLOAB(19858620); Aldehyde dehydrogenase (NAD+) (Clostridium acetobutylicum ATCC 824) gi|15004865|ref|NP_{—}149325.1|(15004865); alcohol dehydrogenase E (Clostridium acetobutylicum) gi|298083|emb|CAA51344.1|(298083); Aldehyde dehydrogenase (NAD+) (Clostridium acetobutylicum ATCC 824) gi|14994477|gb|AAK76907.1|AE001438_{—}160(14994477); aldehyde/alcohol dehydrogenase (Clostridium acetobutylicum) gi|12958626|gb|AAK09379.1|AF321779_{—}1(12958626); Aldehyde-alcohol dehydrogenase, ADHE1 (Clostridium acetobutylicum ATCC 824) gi|15004739|ref|NP_{—}149199.1|(15004739); Aldehyde-alcohol dehydrogenase, ADHE1 (Clostridium acetobutylicum ATCC 824) gi|14994351|gb|AAK76781.1|AE001438_{—}34(14994351); aldehyde-alcohol dehydrogenase E (Clostridium perfringens str. 13) gi|18311513|ref|NP_{—}563447.1|(18311513); aldehyde-alcohol dehydrogenase E (Clostridium perfringens str. 13) gi|18146197|dbj|BAB82237.1|(18146197), each sequence associated with the accession number is incorporated herein by reference in its entirety.

Crotonyl-coA reductase catalyzes the reduction of crotonyl-CoA to butyryl-CoA. Depending upon the organism used a heterologous Crotonyl-coA reductase can be engineered for expression in the organism. Alternatively, a native Crotonyl-coA reductase can be overexpressed. Crotonyl-coA reductase is encoded in S. coelicolor by ccr. CCR homologs and variants are known. For examples, such homologs and variants include, for example, crotonyl CoA reductase (Streptomyces coelicolor A3(2)) gi|21224777|ref|NP_{—}630556.1|(21224777); crotonyl CoA reductase (Streptomyces coelicolor A3(2)) gi|415-4068|emb|CAA22721.1|(415-4068); crotonyl-CoA reductase (Methylobacterium sp. 4-46) gi|168192678|gb|ACA14625.1|(168192678); crotonyl-CoA reductase (Dinoroseobacter shibae DFL 12) gi|159045393|ref|YP_{—}001534187.1|(159045393); crotonyl-CoA reductase (Salinispora arenicola CNS-205) gi|159039522|ref|YP_{—}001538775.1|(159039522); crotonyl-CoA reductase (Methylobacterium extorquens Pa1) gi|163849740|ref|YP_{—}001637783.1|(163849740); crotonyl-CoA reductase (Methylobacterium extorquens Pa1) gi|163661345|gb|ABY28712.1|(163661345); crotonyl-CoA reductase (Burkholderia ambifaria AMMD) gi|115360962|ref|YP_{—}778099.1|(115360962); crotonyl-CoA reductase (Parvibaculum lavamentivorans DS-1) gi|154252073|ref|YP_{—}001412897.1|(154252073); Crotonyl-CoA reductase (Silicibacter sp. TM1040) gi|99078082|ref|YP_{—}611340.1|(99078082); crotonyl-CoA reductase (Xanthobacter autotrophicus Py2) gi|154245143|ref|YP_{—}001416101.1|(154245143); crotonyl-CoA reductase (Nocardioides sp. JS614) gi|119716029|ref|YP_{—}922994.1|(119716029); crotonyl-CoA reductase (Nocardioides sp. JS614) gi|119536690|gb|ABL81307.1|(119536690); crotonyl-CoA reductase (Salinispora arenicola CNS-205) gi|157918357|gb|ABV99784.1|(157918357); crotonyl-CoA reductase (Dinoroseobacter shibae DFL 12) gi|157913153|gb|ABV94586.1|(157913153); crotonyl-CoA reductase (Burkholderia ambifaria AMMD) gi|115286290|gb|ABI91765.1|(115286290); crotonyl-CoA reductase (Xanthobacter autotrophicus Py2) gi|154159228|gb|ABS66444.1|(154159228); crotonyl-CoA reductase (Parvibaculum lavamentivorans DS-1) gi|154156023|gb|ABS63240.1|(154156023); crotonyl-CoA reductase (Methylobacterium radiotolerans JCM 2831) gi|170654059|gb|ACB23114.1|(170654059); crotonyl-CoA reductase (Burkholderia graminis C4D1M) gi|170140183|gb|EDT08361.1|(170140183); crotonyl-CoA reductase (Methylobacterium sp. 4-46) gi|168198006|gb|ACA19953.1|(168198006); crotonyl-CoA reductase (Frankia sp. EAN1pec) gi|158315836|ref|YP_{—}001508344.1|(158315836), each sequence associated with the accession number is incorporated herein by reference in its entirety.

Culture conditions suitable for the growth and maintenance of a recombinant microorganism provided herein are described in the Examples below. The skilled artisan will recognize that such conditions can be modified to accommodate the requirements of each microorganism. Appropriate culture conditions useful in producing a 1-butanol product comprise conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

In one embodiment a microorganism of the disclosure can be characterized as an E. coli comprising rrnBT14DlacZWJ16 hsdR514 DaraBADAH33 DrhaBADLD78 (with F′ transduced from XL-1 blue to supply laciq), Δadh, Δldh, Δfrd polynucleotide, operon or subunit and containing a PJCL50 and pJCL60 plasmid comprising an thl-adhE2, crt-bcd-etfAB-hbd polynucleotide, under the control of the PLlacO1 and an ampicillin resistance gene.

It is understood that a range of microorganisms can be modified to include a recombinant metabolic pathway suitable for the production of n-butanol. It is also understood that various microorganisms can act as “sources” for genetic material encoding target enzymes suitable for use in a recombinant microorganism provided herein. The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the procaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ([NaCl]); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10)Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “recombinant microorganism” and “recombinant host cell” are used interchangeably herein and refer to microorganisms that have been genetically modified to express or over-express endogenous polynucleotides, or to express non-endogenous sequences, such as those included in a vector. The polynucleotide generally encodes a target enzyme involved in a metabolic pathway for producing a desired metabolite as described above, but may also include protein factors necessary for regulation or activity or transcription. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified but which does not express or over-express a target enzyme e.g., an enzyme involved in the biosynthetic pathway for the production of a desired metabolite such as n-butanol.

For example, a wild-type microorganism can be genetically modified to express or over express a first target enzyme such as thiolase. This microorganism can act as a parental microorganism in the generation of a microorganism modified to express or over-express a second target enzyme e.g., hydroxybutyryl-CoA dehydrogenase. In turn, the microorganism modified to express or over express e.g., thiolase and hydroxybutyryl-CoA dehydrogenase can be modified to express or over express a third target enzyme e.g., crotonase.

Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme in to a parental microorganism.

In another embodiment, a method of producing a recombinant microorganism that converts a suitable carbon substrate to n-butanol is provided. The method includes transforming a microorganism with one or more recombinant polynucleotides encoding polypeptides that include keto thiolase or acetyl-CoA acetyltransferase activity, hydroxybutyryl CoA dehydrogenase activity, crotonase activity, crotonyl-CoA reductase or butyryl-CoA dehydrogenase activity, and alcohol dehydrogenase activity.

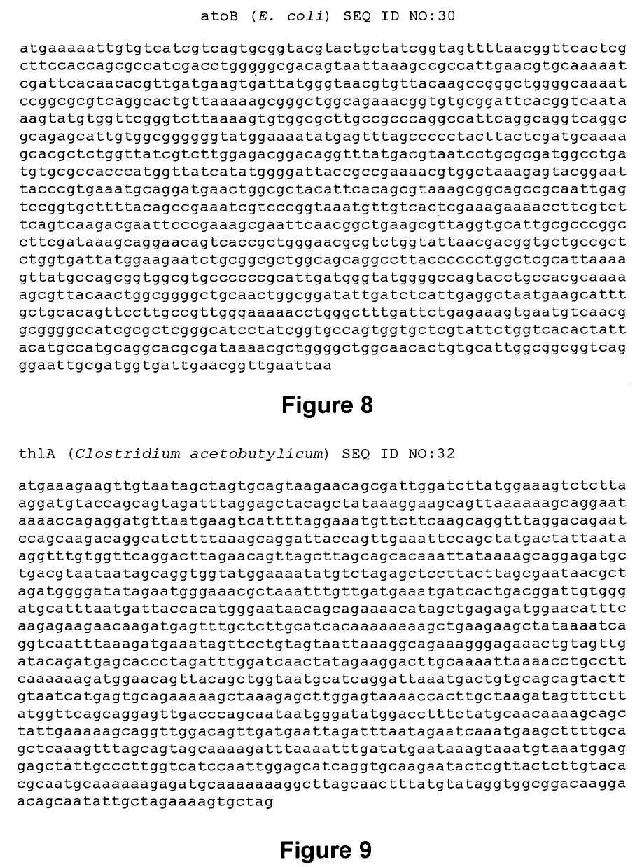

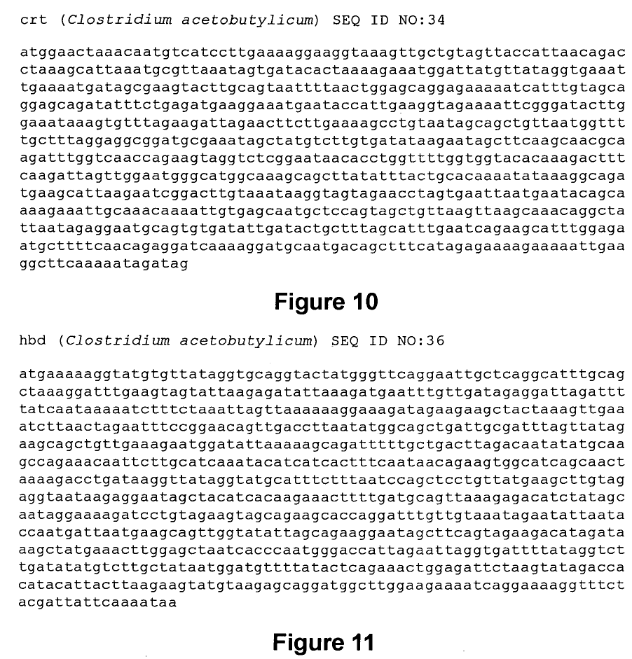

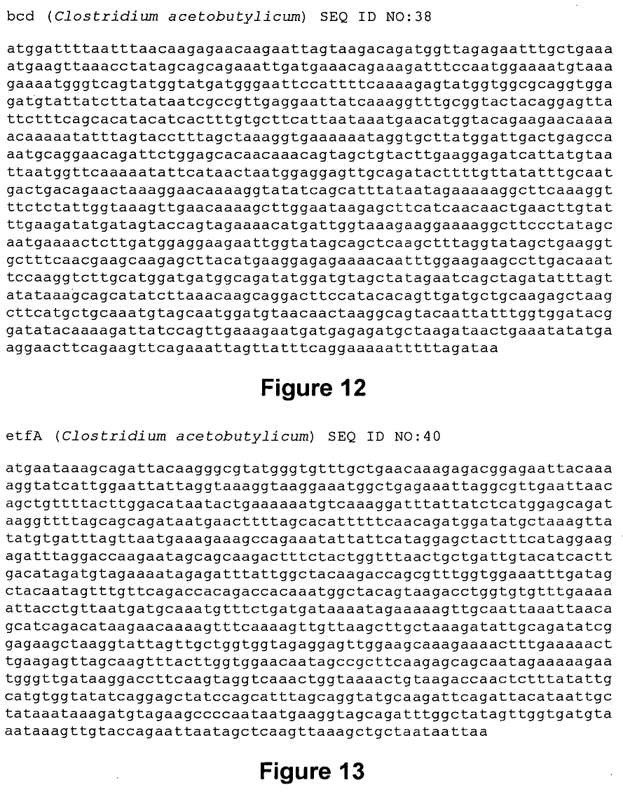

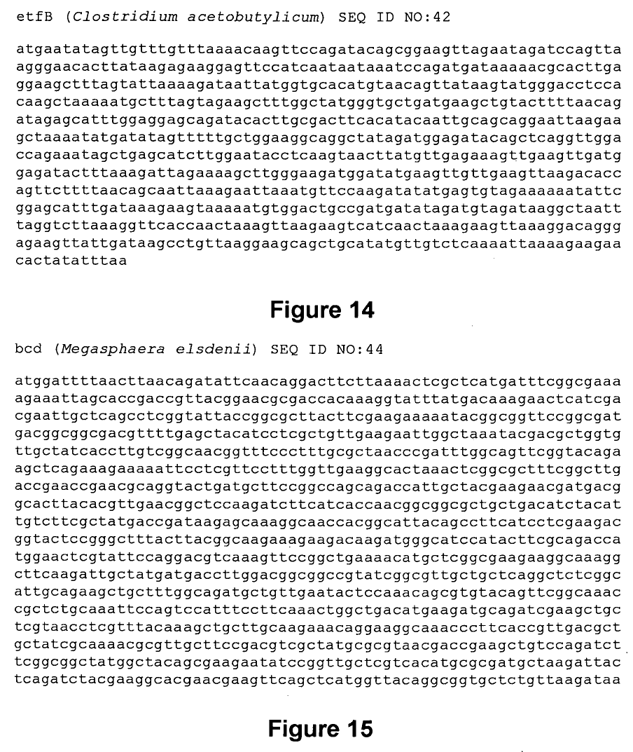

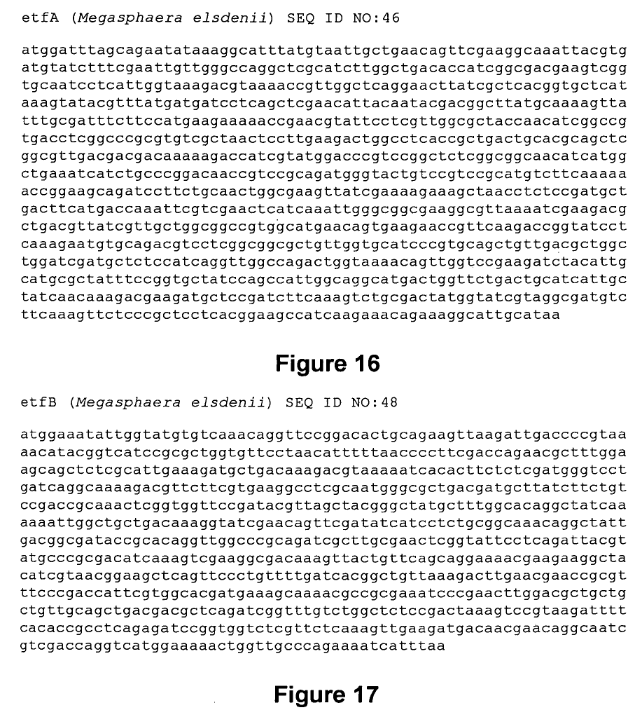

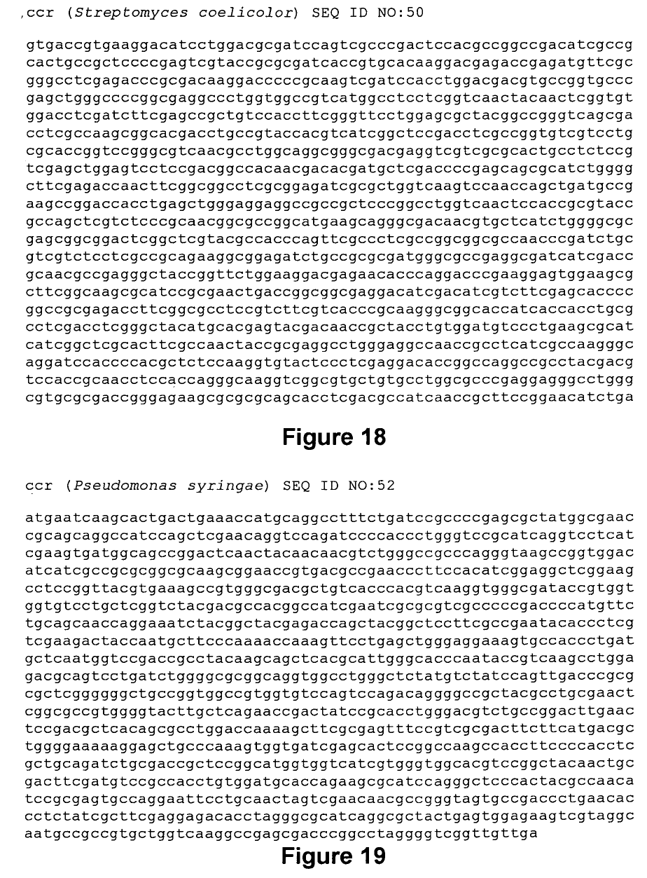

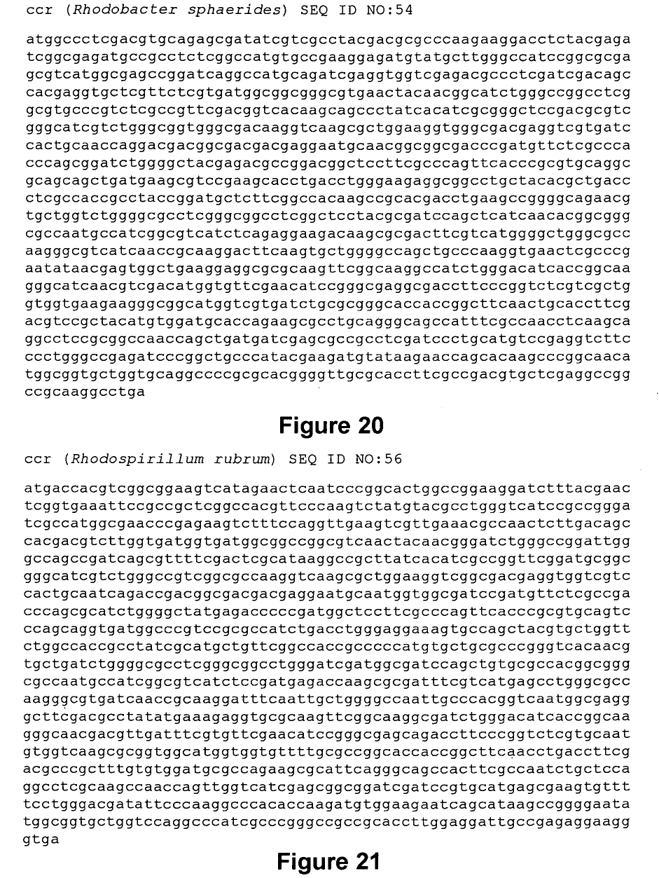

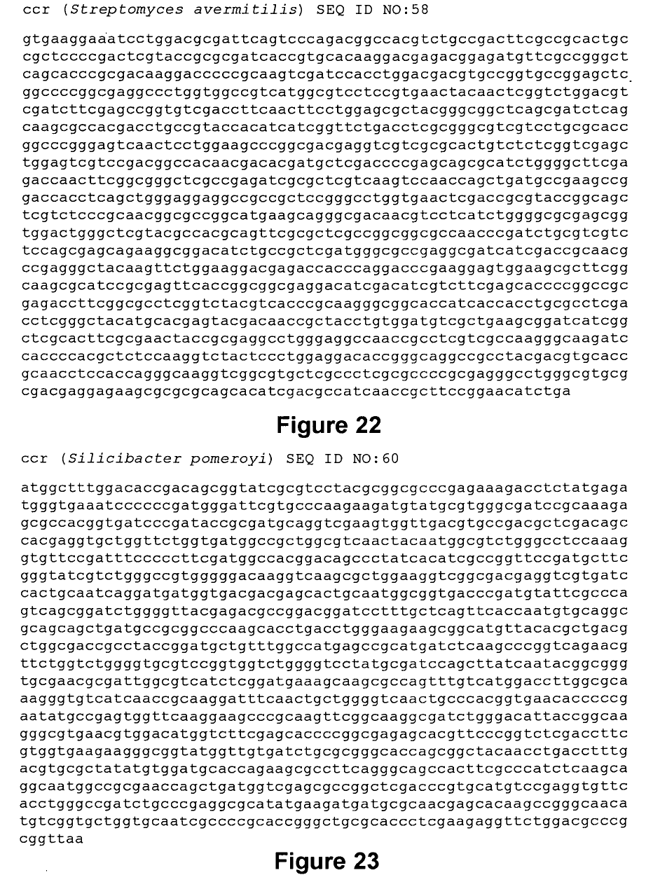

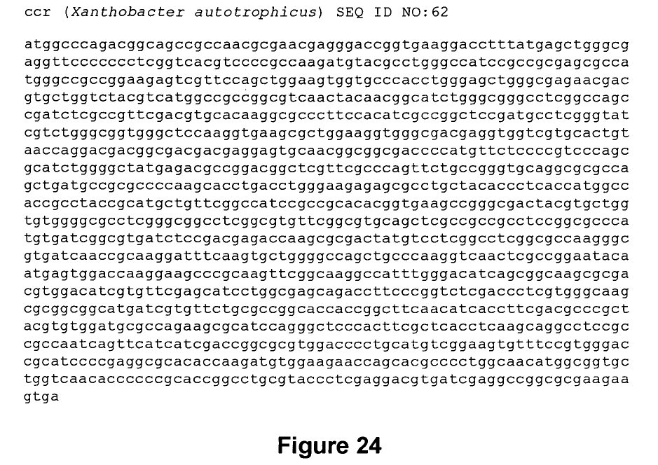

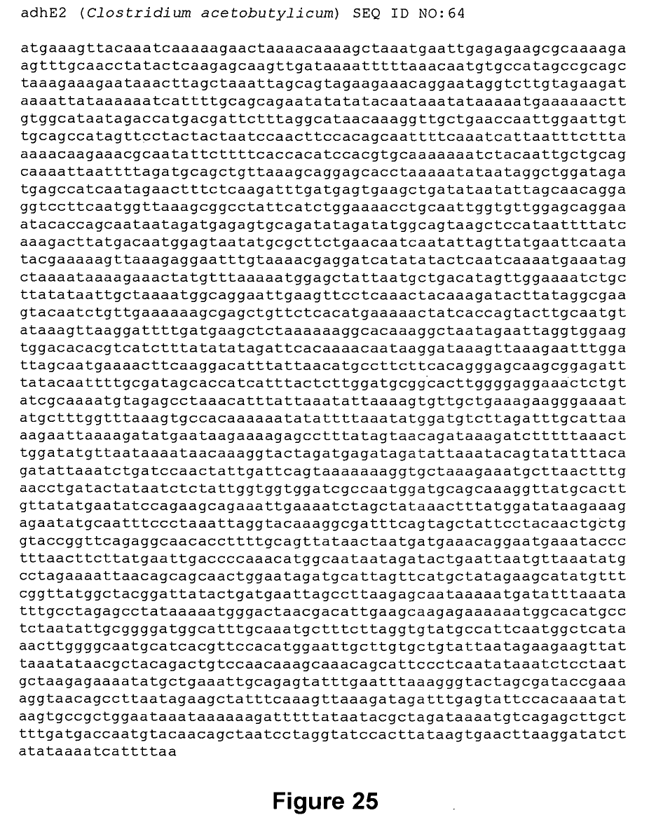

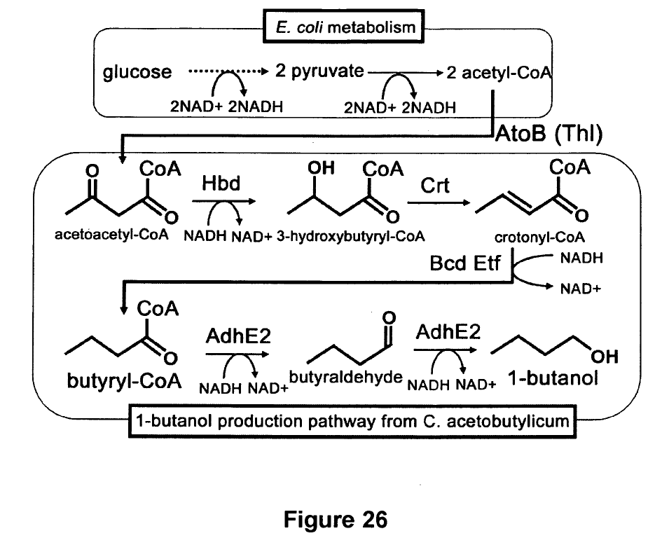

Polynucleotides that encode enzymes useful for generating metabolites (e.g., keto thiolase, acetyl-CoA acetyltransferase, hydroxybutyryl-CoA dehydrogenase, crotonase, crotonyl-CoA reductase, butyryl-CoA dehydrogenase, alcohol dehydrogenase (ADH)) including homologs, variants, fragments, related fusion proteins, or functional equivalents thereof, are used in recombinant nucleic acid molecules that direct the expression of such polypeptides in appropriate host cells, such as bacterial or yeast cells. FIGS. 8 through 25 provide exemplary polynucleotide sequences encoding polypeptides useful in the methods described herein. It is understood that the addition of sequences which do not alter the encoded activity of a nucleic acid molecule, such as the addition of a non-functional or non-coding sequence, is a conservative variation of the basic nucleic acid.

The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

A “protein” or “polypeptide”, which terms are used interchangeably herein, comprises one or more chains of chemical building blocks called amino acids that are linked together by chemical bonds called peptide bonds. An “enzyme” means any substance, preferably composed wholly or largely of protein, that catalyzes or promotes, more or less specifically, one or more chemical or biochemical reactions. The term “enzyme” can also refer to a catalytic polynucleotide (e.g., RNA or DNA).

A “native” or “wild-type” protein, enzyme, polynucleotide, gene, or cell, means a protein, enzyme, polynucleotide, gene, or cell that occurs in nature.

It is understood that a polynucleotide described above include “genes” and that the nucleic acid molecules described above include “vectors” or “plasmids.” For example, a polynucleotide encoding a keto thiolase can comprise an atoB gene or homolog thereof, or an fadA gene or homolog thereof. Accordingly, the term “gene”, also called a “structural gene” refers to a polynucleotide that codes for a particular polypeptide comprising a sequence of amino acids, which comprise all or part of one or more proteins or enzymes, and may include regulatory (non-transcribed) DNA sequences, such as promoter region or expression control elements, which determine, for example, the conditions under which the gene is expressed. The transcribed region of the gene may include untranslated regions, including introns, 5′-untranslated region (UTR), and 3′-UTR, as well as the coding sequence. The term “polynucleotide,” “nucleic acid” or “recombinant nucleic acid” refers to polynucleotides such as deoxyribonucleic acid (DNA), and, where appropriate, ribonucleic acid (RNA). The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate preferred embodiments of the disclosure.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form.

The disclosure provides methods for the heterologous expression of one or more of the biosynthetic genes or polynucleotides involved in n-butanol biosynthesis and recombinant DNA expression vectors useful in the method. Thus, included within the scope of the disclosure are recombinant expression vectors that include such nucleic acids. The term expression vector refers to a polynucleotide that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are preferred and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

Due to the inherent degeneracy of the genetic code, other nucleic acid sequences which encode substantially the same or a functionally equivalent amino acid sequence can also be used to clone and express the polynucleotides encoding such enzymes. As previously noted, the term “host cell” is used interchangeably with the term “recombinant microorganism” and includes any cell type which is suitable for producing e.g., n-butanol and susceptible to transformation with a nucleic acid construct such as a vector or plasmid.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions.

In another embodiment, a method for producing n-butanol is provided. The method includes culturing a recombinant microorganism as provided herein in the presence of a suitable carbon substrate and under conditions suitable for the conversion of the substrate to n-butanol.

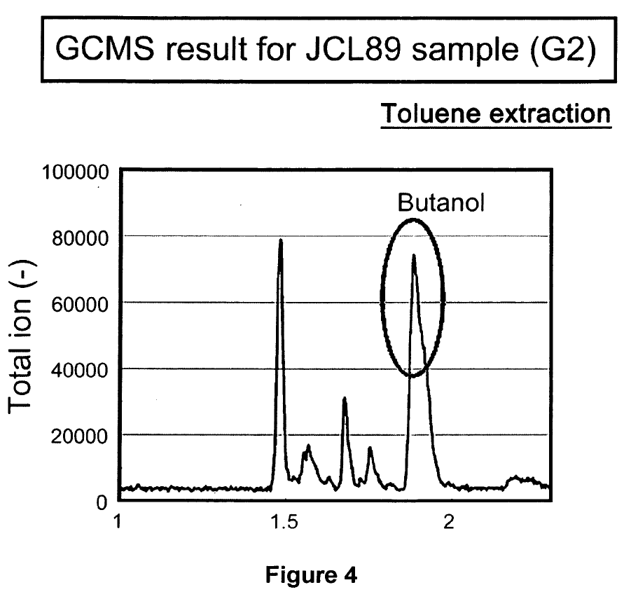

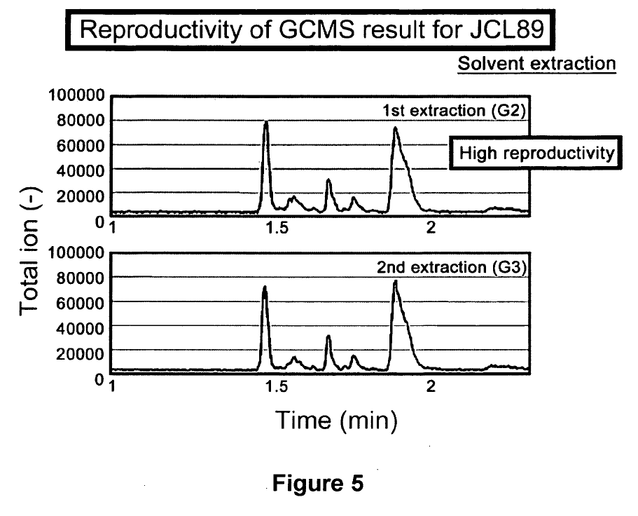

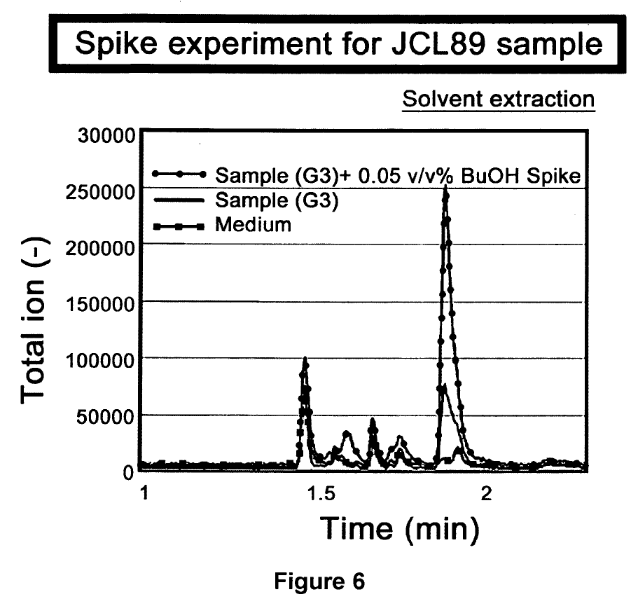

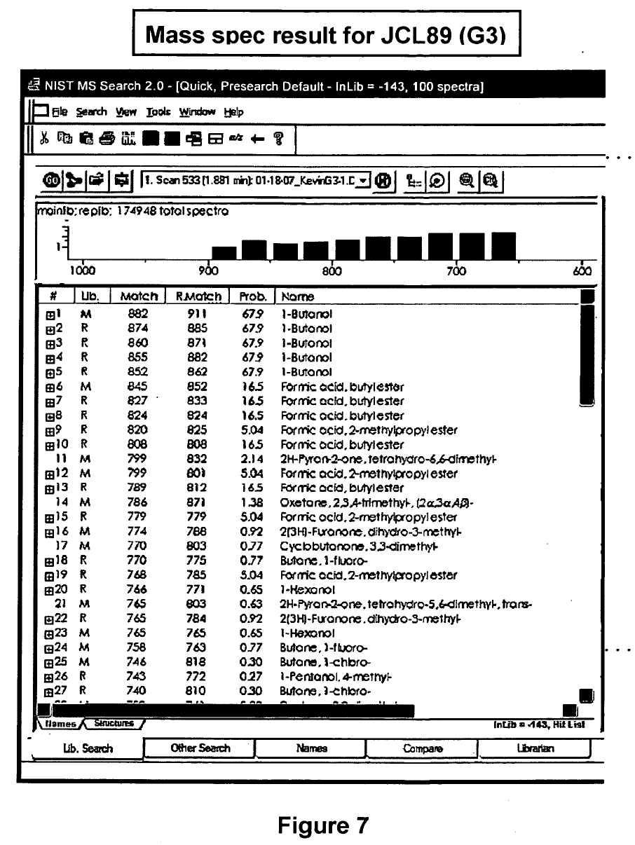

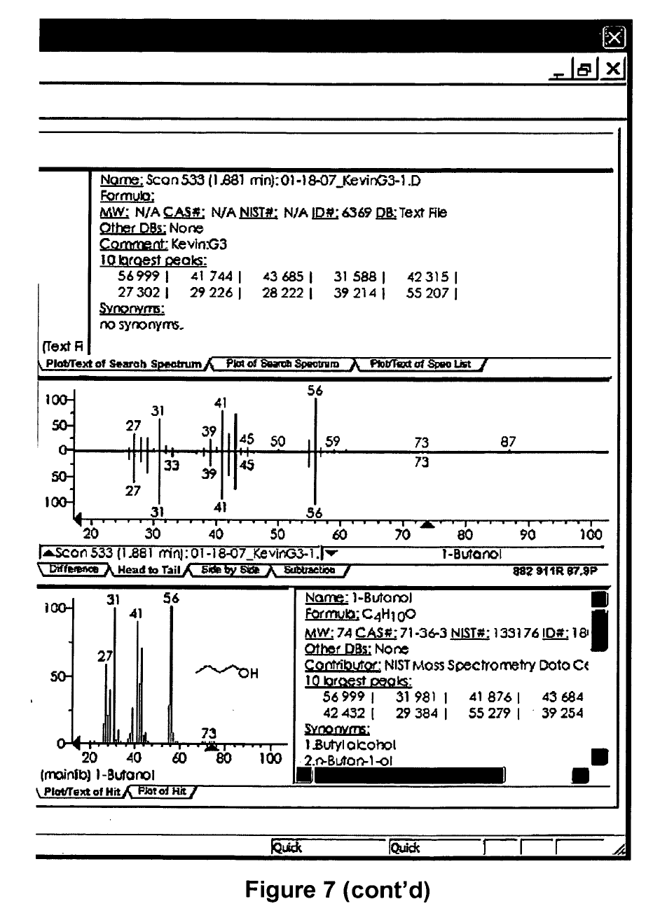

The butanol produced by a microorganism provided herein can be detected by any method known to the skilled artisan. Such methods include mass spectrometry as described in more detail below and as shown in FIGS. 4-6.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; Barringer et al. (1990) Gene 89: 117; and Sooknanan and Malek (1995) Biotechnology 13: 563-564.

Improved methods for cloning in vitro amplified nucleic acids are described in Wallace et al., U.S. Pat. No. 5,426,039.

Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The invention is illustrated in the following examples, which are provided by way of illustration and are not intended to be limiting.

### EXAMPLES

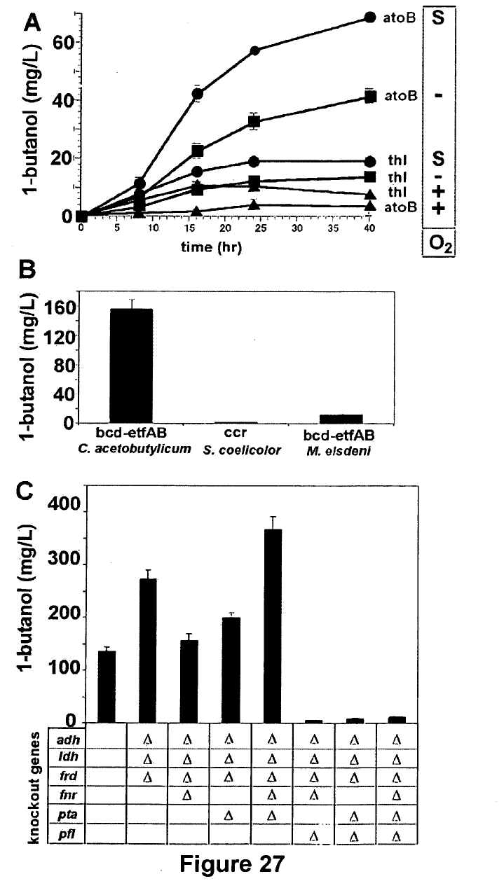

In C. acetobutylicum, the 1-butanol pathway branches off to produce acetone and butyrate. In the present studies, various genes for 1-butanol production were transferred. These genes (thl, hbd, crt, bcd, etfAB, adhE2) were cloned and expressed in E. coli using two plasmids (pJCL50 and pJCL60, see Table 1) under the control of the IPTG-inducible PLlacO1 promoter. The activity of these gene products were successfully detected by enzyme assays except bcd and etfAB which code for butyryl-CoA dehydrogenase (Bcd) and an electron transfer flavoprotein (Etf). The activity of butyryl-CoA dehydrogenase was not conclusively demonstrated using crude extract from cells that expressed bcd and etfAB. Despite the inconclusive demonstration of Bcd activity, the expression of this synthetic pathway produced 13.9 mg/L of 1-butanol under anaerobic conditions (see FIG. 27, Panel A). In contrast to the suspected oxygen sensitivity, a slight increase in the oxygen level increased the production of 1-butanol, suggesting that the NADH produced anaerobically was insufficient to supply for 1-butanol production. In a completely aerobic condition, on the other hand, E. coli consumes both acetyl-CoA and NADH in TCA cycle and respiration, and thus likely contributes to the decreased 1-butanol production (see FIG. 27, Panel A).

In addition to the C. acetobutylicum thiolase (coded by thl), the E. coli atoB gene product (acetyl-CoA acetyltransferase) was determined to catalyze the first reaction from acetyl-CoA to acetoacetyl-CoA. The production of 1-butanol increased more than 3-fold (see FIG. 27, Panel A). To determine whether homologues and isoenzymes of Bcd from other organisms would be more effective in E. coli, bcd and etfAB were expressed from Megasphaera elsdenii and ccr from Streptomyces coelicolor, which encodes a crotonyl-CoA reductase (Ccr) that does not require an Etf for activity, in place of their counterparts from C. acetobutylicum. The activity of S. coelicolor Ccr, but not M. elsdenii Bcd, was detected conclusively by enzyme assays using crude extracts. However, the M. elsdenii and S. coelicolor genes led to a lower production of 1-butanol in E. coli (FIG. 27, Panel B). It is understood that alternative genes from other organisms may be used to improve 1-butanol production in E. coli.

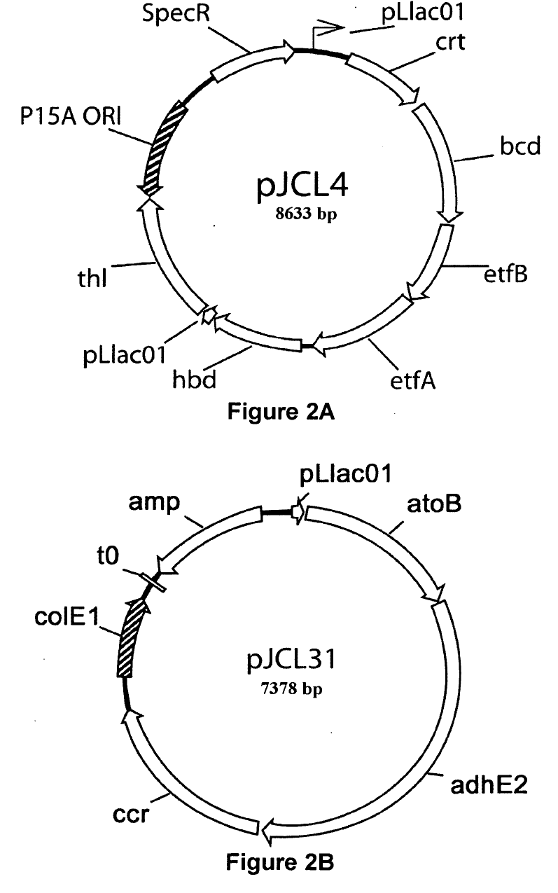

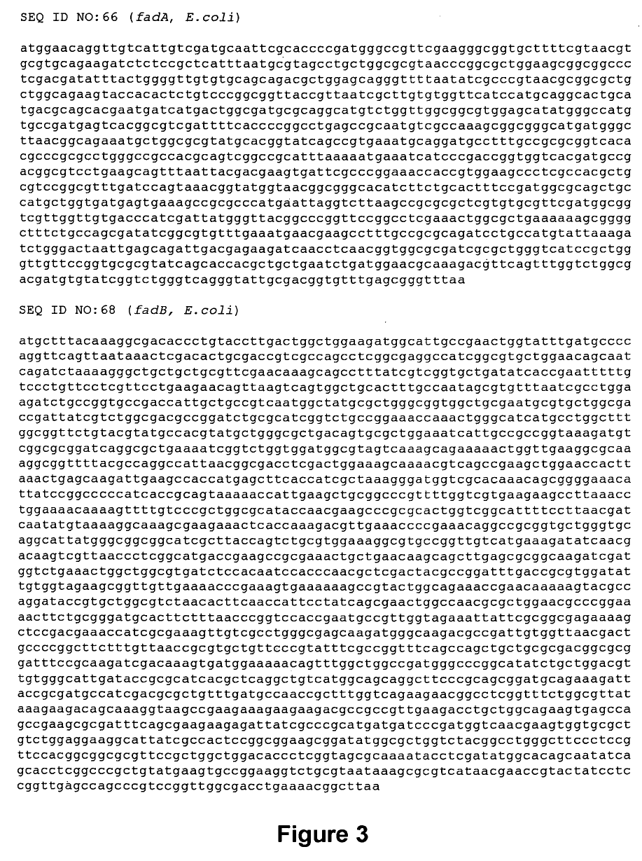

To further improve 1-butanol production, the host pathways that compete with the 1-butanol pathway for acetyl-CoA and NADH were deleted. FIG. 2, Panel C shows that deletion of ldhA, adhE, and frdBC from WT, complete with the 1-butanol production pathway (JCL184), doubled the production of 1-butanol by significantly reducing the amount of lactate, ethanol, and succinate produced (see Table 2 below). The decision to knock out the native adhE in E. coli and replace it with adhE2 from C. acetobutylicum was based on the relative affinities of each enzyme towards acetyl-CoA and butyryl-CoA. While the activity of the adhE2 gene product for butyryl-CoA (0.08 μmol min-1 (mg protein)-1) is not much higher than that of the adhE gene product (0.05), its activity for acetyl-CoA (0.05) is four times less than that of the adhE encoded enzyme (0.22) for the same substrate. This ratio favors adhE2 over adhE for 1-butanol production.

Although the deletions in JCL184 resulted in the decrease of most fermentation products, a significant amount of acetate was produced. To further increase 1-butanol production, pta was deleted. While acetate production was decreased considerably, this strain (JCL275) led to a lower production of 1-butanol.

The deletion of pf1B (JCL168, JCL171 and JCL260) nearly abolished 1-butanol production, indicating that pyruvate-formate lyase (Pfl) was responsible for the production of acetyl-CoA from pyruvate under the experimental condition (see FIG. 27, Panel C). The use of Pfl results in the loss of the reducing equivalent to formate. It is therefore desirable to use the pyruvate dehydrogenase complex (PDHc) for the production of 1-butanol, since the reducing power is stored in NADH rather than formate. To achieve elevated expression of PDHc, fnr was deleted. Fnr encodes an anaerobic regulator that represses the expression of PDHc genes during anaerobic growth. The deletion of fnr from JCL184 decreased 1-butanol production. However, when both pta and fnr were deleted (JCL187), production of 1-butanol improved nearly three-fold over wild type levels (373 mg/L). This improvement in 1-butanol production was accompanied by an increase of ethanol production to wild type levels. The mechanism for the elevated 1-butanol production in the strain appears to be complex and requires further investigation.

Referring to FIG. 1, the conversion from acetyl-CoA to acetoacetyl-CoA was achieved by over-expression of either E. coli atoB or Clostridium thlA. The structural organization and regulation of the genes involved in short-chain fatty acid degradation in E. coli, referred to as the “ato” system, have been studied by a combination of classic genetic and recombinant DNA techniques. In general, the atoB gene encodes a keto thiolase. The ato regulatory locus has been designated atoC. Increased production of acetoacetyl-CoA by the increased expression of the E. coli keto thiolase (atoB) can increase the down-stream production of intermediates required for the synthesis of n-butanol.

In addition, acetyl-CoA acetyltransferase activity encoded by the thlA gene from Clostridium acetobutylicum can be used in this step of the pathway to increase production of acetoacetyl-CoA.

Genes encoding thiolase enzymes can be obtained from a range of bacteria, mammals and plants. At least five different thiolases have been identified in E. coli. Two of these thiolases are encoded by previously identified genes, fadA and atoB, whereas three others are encoded by open reading frames that can be expressed using any suitable expression system.

Referring again to FIG. 1, the second (2) and third (3) steps of the pathway, from acetoacetyl-CoA to crotonyl-CoA was achieved using the hbd and crt genes from Clostridium acetobutylicum. The C. acetobutylicum locus involved in butyrate fermentation encodes 5 enzymes/proteins: crotonase (crt), butyryl-CoA dehydrogenase (bcd), 2 ETF proteins for electron transport (etfA and etfB), and 3-hydroxybutyryl-CoA dehydrogenase (hbd) (Boynton et al., J. Bacteriol. 178: 3015 (1996), which is incorporated herein by reference in its entirety). Another microorganism from which these genes have been isolated is Thermoanaerobacterium thermosaccharolyticum. Hbd and crt have been isolated from C. difficile as well (Mullany et al., FEMS Microbiol. Lett. 124: 61 (1994), which is incorporated herein by reference in its entirety). 3-hydroxybutyryl-CoA dehydrogenase activity has been detected in Dastricha ruminatium (Yarlett et al., Biochem. J. 228: 187 (1995)), Butyrivibrio fibrisolvens (Miller & Jenesel, J. Bacteriol., 138: 99 (1979)), Treponema phagedemes (George & Smibert, J. Bacteriol., 152: 1049 (1982)), Acidaminococcus fermentans (Hartel & Buckel, Arch. Microbiol., 166: 350 (1996)), Clostridium kluyveri (Madan et al., Eur. J. Biochem., 32: 51 (1973)), Syntrophosphora bryanti (Dong & Stams, Antonie van Leeuwenhoek, 67: 345 (1995), each of which is incorporated herein by reference in its entirety); crotonase activity has been detected in Butyrivibrio fibrisolvens (Miller & Jenesel, J. Bacteriol., 138: 99 (1979), which is incorporated herein by reference in its entirety); and butyryl-CoA dehydrogenase activity has been detected in Megasphaera elsdenii (Williamson & Engel, Biochem. J., 218: 521 (1984)), Peptostreptococcus elsdenii (Engel & Massay, Biochem. J., 1971, 125: 879), Syntrophosphora bryanti (Dong & Stams, Antonie van Leeuwenhoek, 67: 345 (1995)), and Treponema phagedemes (George & Smibert, J. Bacteriol., 152: 1049 (1982), each of which is incorporated herein by reference in its entirety).

Referring again to FIG. 1, the fourth (4) step, the conversion of crotonyl-CoA to butyryl-CoA was achieved using Streptomryces coelicolor or Streptomryces collinus ccr gene (encoding crotonyl-CoA reductase), or Megasphaera elsdenii bcd gene (encoding butyryl-CoA dehydrogenase). As previously noted, the pathway from acetyl-CoA to butyryl-CoA is best understood in Clostridum acetobutylicum, which produces high levels of butanol. However, homologous polynucleotides encoding polypeptides useful in the pathway have been cloned from various sources. For example, at least one counterpart of each gene has been shown to be present in the genome of Streptomyces coelicolor. Genes for the entire pathway from acetyl-CoA to butyryl-CoA are thus accessible.

As shown in the present studies crotonyl CoA can be converted to butyryl CoA by the enzyme crotonyl CoA reductase encoded by the ccr gene. The ccr gene can be isolated from Streptomryces coelicolor, Streptomryces collinus, or other host cells. The butyryl CoA dehydrogenase (bcd) gene can be obtained from Clostridium acetobutylicum or Mycobacterium tuberculosis (e.g., fadE25). The last two steps (see FIG. 1 at 5 and 6), from butyryl-CoA to n-butanol was achieved using the adhE2 gene from Clostridium acetobutylicum.

The genes can be cloned in to any suitable vector. Table 1 (see below) provides a list of exemplary strains and constructs suitable for use as vectors. EC=Escherichia coli, ME=Megasphaera elsdenii, SC=Streptomryces coelicolor. The other genes are from Clostridium acetobutylicum.

The two plasmids, pJCL4 and pJCL31 were transformed into an E. coli host JCL88 and the resulting transformants were grown in M9 medium containing 40 g/l of glucose at 37° C. under shaking. After 24 hours, the culture broth was sampled for product analysis using GC-mass spectrometer. The results show that n-butanol was produced to a level approximately 0.05 g/L (see chromatogram e.g., in FIG. 4).

In constructing the strains provided herein and shown in FIG. 1, one may desire to determine accurately the levels of metabolic intermediates (e.g., acetoacetyl-CoA, crotonyl-CoA, etc) in cells grown under various conditions. Various methods for determining the presence of such intermediates are available and known to the skilled artisan. For example, the extraction of metabolic intermediates from cells and their subsequent partial purification by HPLC analysis can be employed. The identities of the intermediates can be confirmed by LC/MS analysis.

As previously noted, Table 1 further provides a list of strains used in the present studies. Gene deletion was facilitated via methods known in the art. BW25113 (rrnB_{T14} ΔlacZ_{WJ16} hsdR514 ΔaraBAD_{AH33} ΔrhaBAD_{LD78}) was used as WT. The adh, ldh, frd, fnr, and pflB sequences were deleted. The pta deletion was made by P1 transduction with JW2294 (Baba et al. Mol. Syst. Biol. (2006), which is incorporated herein by reference in its entirety) as the donor. F′ was transferred from XL⁻1 blue to supply lacI^{q}.

TABLE 1
Strains and Plasmids Used

| | | |
|---|---|---|
| Name | Relevant Genotype | Reference |

Strains

| | | |
|---|---|---|
| BW25113 | rrnB_{T14} DlacZWJ16 hsdR514 DaraBAD_{AH33} DrhaBAD_{LD78} | Datsenko and |
| | | Wanner, 2000 |
| XL-1 Blue | recA1 endA1 gyrA96 thi-1 hsdR17 supE44 relA1 lac | Stratagene |
| | [F′ proAB lacI^{q}ZΔM15 Tn10 (Tet^{R})] | |
| JCL16 | BW25113/F′ [traD36, proAB+, lacIq ZΔM15] | |
| JCL88 | JCL16 ΔadhE, ldhA, frdBC, fnr, pta | |
| JCL166 | JCL16 ΔadhE, ldhA, frdBC | |
| JCL167 | JCL16 ΔadhE, ldhA, frdBC, fnr | |
| JCL168 | JCL16 ΔadhE, ldhA, frdBC, fnr, pflB | |
| JCL170 | JCL16 ΔadhE, ldhA, frdBC, fnr, pta, pntA | |
| JCL171 | JCL16 ΔadhE, ldhA, frdBC, pta, pflB | |
| JCL184 | JCL166/pJCL17/pJCL60 | |
| JCL185 | JCL167/pJCL17/pJCL60 | |
| JCL186 | JCL168/pJCL17/pJCL60 | |
| JCL187 | JCL88/pJCL17/pJCL60 | |
| JCL190 | JCL171/pJCL17/pJCL60 | |
| JCL191 | JCL16/pJCL17/pJCL60 | |
| JCL198 | JCL16/pJCL50/pJCL60 | |
| JCL230 | JCL88/pJCL17/pJCL63 | |
| JCL235 | JCL88/pJCL17/pJCL74 | |
| JCL260 | JCL16 ΔadhE, ldhA, frdBC, fnr, pta, pflB | |
| JCL262 | JCL260/pJCL17/pJCL60 | |
| JCL274 | JCL16 ΔadhE, ldhA, frdBC, pta | |
| JCL275 | JCL274/pJCL17/pJCL60 | |

Plasmids

| | | |
|---|---|---|
| pZE12-luc | ColE1 ori; Amp^{R}; P_{L}lacO₁: luc(VF) | Lutz and Bujard, 1997 |
| pZE21-MCS1 | ColE1 ori; Kan^{R}; P_{Ltet}O₁: MCS1 | Lutz and Bujard, 1997 |
| pACYC184 | pl5A ori; Cm^{R}; Tet^{R} | New England Biolabs |
| pJCL17 | ColE1 ori; Amp^{R}; P_{L}lacO₁: atoB(EC)-adhE2(CA) | |
| pJCL50 | ColE1 ori; Amp^{R}; P_{L}lacO₁: thl(CA)-adhE2(CA) | |
| pJCL60 | p15A ori; Spec^{R}; P_{L}lacO₁: crt(CA)-bcd(CA)-etfAB(CA)-hbd(CA) | |
| pJCL63 | p15A ori; Cm^{R}; P_{L}lacO₁: crt-bcd(ME)-ccr(SC)-hbd(CA) | |
| pJCL74 | p15A ori; Cm^{R}; P_{L}lacO₁: crt-bcd(ME)-etfAB(ME)-hbd(CA) | |

Referring to FIG. 2A, various plasmids were constructed according to the following exemplary protocols:

To clone crt, bcd, etfAB, hbd, genomic DNA of Clostridium acetobutylicum ATCC824 (ATCC) was used as a PCR template with a pair of primers designated crtXmaIf and hbdSacIr (fragment 1). To make a plasmid backbone, pJRB1-rc (pACYC derivative, specr, araC, PBAD) was used. Fragment 1 and the backbone were digested with XmaI and SacI and ligated, creating pJCL2. To replace PBAD with PLlacO1, pZE12-luc was used as PCR template with primers A46 and A47. PCR products were digested with NcoI and XmaI and ligated into the matching sites of pJCL2 to create pJCL60.

To replace PL-tetO1 of pZE21-MCS1 with PL-lacO1, pZE12-luc was digested with AatII and Acc65I. The shorter fragment was purified and cloned into the corresponding sites of pZE21-MCS1 to create pSA40. crt was amplified from C. acetobutylicum ATCC824 genomic DNA using primers A85 and A86. The PCR product was digested with Acc65I and SalI and cloned into pSA40 cut with the same enzymes, creating pJCL33. pJCL35 was created by amplifying the hbd gene fragment from C. acetobutylicum genomic DNA with primers A89 and A90, digesting the PCR fragment with XmaI and MluI, and ligating the product into the corresponding sites of pJCL33. The ColE1 origin was replaced with p15A by digesting pZA31-luc with AatII and AvrII. The smaller fragment was purified and cloned into pJCL35 digested with the same enzymes, creating pJCL37. To eliminate a point mutation in the crt gene of pJCL37, crt was amplified and digested as described previously and ligated into the corresponding sites of pJCL37 to create pJCL66. The S. coelicolor ccr gene was amplified from genomic DNA using primers A87 and A88. The product was digested with SalI and XmaI, and cloned into the same sites of pJCL66 to create pJCL63. M. elsdenii bcd and etfBA was amplified from a synthesized template (Epoch Biolabs, Sugar Land, Tex.) using primers MegBcd-op-fwd and MegBcd-op-rev. The PCR product was digested with XhoI and XmaI and ligated into the SalI and XmaI sites of pJCL66 to create pJCL74.

The C. acetobutylicum ATCC824 thl was amplified from genomic DNA using primers thlAcc65I and thlSphIr. The product was digested with Acc65I and SphI and ligated into the Acc65I and SphI sites of pZE12-luc to create pJCL43. pJCL43 was then digested with SpeI and SphI, and the larger fragment was purified and cloned into the larger fragment created by digestion with SpeI and SphI of pJCL17, creating pJCL50.

To replace PBAD with PLlacO1, pZE12-luc was used as PCR template with a pair of primers designated A46 and A47 (fragment 3). pJCL3 was used as a plasmid backbone. Fragment 3 and the backbone were digested with NcoI and XmaI and ligated, creating pJCL4.

To clone atoB, genomic DNA of Escherichia coli MG1655 was used as PCR template with a pair of primers designated atoBAcc65I and atoBSphI. PCR products were digested with Acc65I and SphI and cloned into pZE12-luc cut with the same enzyme, creating pJCL16. AdhE2 was amplified from the pSOL1 megaplasmid in a total DNA extract of C. acetobutylicum DNA using adhE2SphIf and adhE2XbaIr. The PCR product was digested with SphI and XbaI and ligated into the same sites of pJCL16 to create pJCL17.

To clone adhE2, pSOL1 in genomic DNA solution of Clostridium acetobutylicum ATCC824 (ATCC) was used as PCR template with a pair of primers designated adhE2SphI and adhE2XbaI. PCR products were digested with SphI and XbaI and cloned into pJCL16 cut with the same enzyme, creating pJCL17.

To clone ccr, genomic DNA of Streptomyces coelicolor was used as PCR template with a pair of primers designated A95 and ccrXbaIr. PCR products were digested with XbaI and cloned into pJCL17, creating pJCL31.

Table 2 provides a list of exemplary byproducts of 1-butanol producing strains.

TABLE 2
Metabolic Byproducts of 1-Butanol Producing Strains

| | Concentration (mM) | | | | | |
|---|---|---|---|---|---|---|
| Strain | Acetate | Ethanol | Formate | Lactate | Succinate | Glucose¹ |
| JCL184 | 15.17 | 3.00 | 23.12 | 5.44 | 0.71 | 30.69 |
| JCL185 | 11.80 | 2.50 | 16.40 | 2.49 | 1.17 | 22.24 |
| JCL186 | 4.86 | 0.50 | 3.46 | 2.91 | 2.52 | 14.10 |
| JCL187 | 1.48 | 7.70 | 20.97 | 2.99 | 1.72 | 42.75 |
| JCL190 | 0.71 | 0.30 | 2.09 | 1.87 | 1.16 | 14.31 |
| JCL191 | 13.48 | 7.60 | 19.54 | 41.77 | 3.35 | 44.88 |
| JCL262 | 0.71 | 0.80 | 3.02 | 2.93 | 2.25 | 18.25 |
| JCL275 | 1.28 | 1.50 | 18.50 | 2.43 | 1.13 | 28.22 |

¹Glucose Consumed

Table 3 (see below) provides a list of exemplary oligonucleotide primers. Table 3 also provides the nucleic acid sequence of each exemplary primer. The sequences provided in Table are useful for initiating and sustaining the amplification of a target polynucleotide. It is understood that alternative sequences are similarly useful for amplifying a target nucleic acid. Accordingly, the methods described herein are not limited solely to the primers described below.

TABLE 3
oligonucleotides

| | | |
|---|---|---|
| | | SEQ ID |
| name | sequence | NO: |

| | | |
|---|---|---|
| adhEfwk0 | ATTCGAGCAGATGATTTACTAAAAAAGTTTA | 1 |
| | ACATTATCAGGAGAGCATTGTGTAGGCTGG | |
| | AGCTGCTTC | |
| adhErvko | CCCAGAAGGGGCCGTTTATGTTGCCAGACAG | 2 |
| | CGCTACTGACATATGAATATCCTCCTTAG | |
| frdBCp1 | GCCGATAAGGCGGAAGCAGCCAATAAGAAGG | 3 |
| | AGAAGGCGAGTGTAGGCTGGAGCTGCTTC | |
| frdBCp2 | GTCAGAACGCTTTGGATTTGGATTAATCATC | 4 |
| | TCAGGCTCCCATATGAATATCCTCCTTAG | |
| ldhAp1 | CTTAAATGTGATTCAACATCACTGGAGAAAG | 5 |
| | TCTTGTGTAGGCTGGAGCTGCTTC | |
| ldhAp2 | ATCTGAATCAGCTCCCCTGGAATGCAGGGGA | 6 |
| | GCGGCAAGACATATGAATATCCTCCTTAG | |
| crtXmaIf | GCGCCCGGGTTAGGAGGATTAGTCATGGAAC | 7 |
| | TAA | |
| hbdSacIr | GGCGAGCTCCCCCATTTGATAATGGGGATTC | 8 |
| | TTG | |
| CAC28731acO1f | AATGATACTTAGATTCAATTGTGAGCGGATA | 9 |
| | ACAATTTCACACAGGAGGTTAGTTAGAATGA | |
| | AAGAAG | |
| Pthlf(-P) | GAATGAAGTTTCTTATGCACAAGTA | 10 |
| ThlClaIr | CAGATCGATCTAGCACTTTTCTAGCAATATT | 11 |
| | GC | |
| A46 | AATAATCCATGGCGTATCACGAGGCCCTTTC | 12 |
| | GTCT | |
| A47 | AATAACCCGGGTCAGTGCGTCCTGCTGATGT | 13 |
| | GCT | |
| atoBAcc65If | CGAGCGGTACCATGAAAAATTGTGTCATCGT | 14 |
| | CAGTG | |
| atoBSphIr | CCGCATGCTTAATTCAACCGTTCAATCACCA | 15 |
| | TC | |
| adhE2SphIf | CCGCATGCAGGAGAAAGGTACCATGAAAGTT | 16 |
| | ACAAATCAAAAAGAACTAAAACAA | |
| adhE2XbaIr | GCGCATCTAGATTAAAATGATTTTATATAGA | 17 |
| | TATCC | |
| A95 | GCTCTAGAAGGAGATATACCATGACCGTGAA | 18 |
| | GGACATCCTGGACG | |
| ccrXbaIr | CTTCTAGATCAGATGTTCCGGAAGCGGTTGA | 19 |
| | TG | |
| thlAcc65If | TCAGGTACCATGAAAGAAGTTGTAATAGCTA | 20 |
| | GTGCAGTA | |
| thlSphIr | TCAGCATGCCTAGCACTTTTCTAGCAATATT | 21 |
| | GCTGTT | |
| A85 | CGAGCGGTACCATGGAACTAAACAATGTCAT | 22 |
| | CCTTG | |
| A86 | ACGCAGTCGACCTATGAAAGCTGTCATTGCA | 23 |
| | TCCTT | |
| A89 | AATAACCCGGGAGGAGATATACCATGAAAAA | 24 |
| | GGTATGTGTTATAGGTG | |
| A90 | CGAGCACGCGTTTATTTTGAATAATCGTAGA | 25 |
| | AACCT | |
| A87 | ACGCAGTCGACAGGAGATATACCATGACCGT | 26 |
| | GAAGGACATCCTGGACG | |
| A88 | AATAACCCGGGTCAGATGTTCCGGAAGCGGT | 27 |
| | TGATG | |
| MegBcd-op-fwd | TAATCTCGAGTAAGGAGAGTGGAACATCATG | 28 |
| | GATT | |
| MegBcd-op-rev | TTAACCCGGGCTTATGCAATGCCTTTCTGTT | 29 |
| | CTT | |

For all experiments, 16 hr precultures in M9 medium (6 g Na₂HPO₄, 3 g KH2PO4, 0.5 g NaCl, 1 g NH₄Cl, 1 mM MgSO₄, 10 mg Vitamin B1 and 0.1 mM CaCl₂ per liter water) containing 2% glucose, 0.1M MOPS and 1000× Trace Metal Mix (27 g FeCl₃.6H₂O, 2 g ZnCl₂.4H₂O, 2 g CaCl₂.2H₂O, 2 g Na₂MoO₄.2H₂O, 1.9 g CuSO₄.5H₂O, 0.5 g H₃BO₃, 100 mL HCl per liter water) were inoculated 1% from an overnight culture in LB and grown at 37° C. in a rotary shaker (250 rpm). For the knockout strain comparisons, 0.1% casamino acids were added to the media. Antibiotics were added appropriately (ampicillin 100 μg/mL, chloroamphenicol 40 μg/mL, spectinomycin 20 μg/mL, kanamycin 30 μg/mL).

For anaerobic growth, precultures were adjusted to OD₆₀₀ 0.4 with 12 mL of fresh medium with appropriate antibiotics and induced with 0.1 mM IPTG. The culture was transferred to a sealed 12 mL glass tube (BD Biosciences, San Jose, Calif.) and the headspace was evacuated. Cultures were shaken (250 rpm) at 37° C. for 8-40 hr. Semi-aerobic cultures were grown similarly, except that 5 mL of fresh medium was added and transferred to the sealed glass tubes without evacuation of the headspace. Aerobic cultures were diluted with 3 mL of fresh media and grown in unsealed capped test tubes.

All restriction enzymes and Antarctic phosphatase was purchased from New England Biolabs (Ipswich, Mass.). The Rapid DNA ligation kit was supplied by Roche (Manheim, Germany). KOD DNA polymerase was purchased from EMD Chemicals (San Diego, Calif.). Oligonucleotides were ordered from Invitrogen (Carlsbad, Calif.).

E. coli genes adhE, ldhA, frdBC, fnr, pflB were deleted by techniques known to the skilled artisan. Phosphate acetyltransferase, encoded by pta, was inactivated by P1 transduction with JW2294 as the donor. F′ was transferred from XL-1 blue (Stratagene) to supply lacIq. All plasmids listed in Table 1 were sequenced to verify the accuracy of the cloning.

Cultures were grown in 50 mL SOB medium in a sealed 50 mL tube at 37° C. in a rotary shaker (250 rpm). At OD₆₀₀ 0.8, cultures were induced with 0.1 mM IPTG and grown for one additional hour before 50 fold concentration in 100 mM Tris-HCl buffer (pH 7.0) and lysing with 0.1 mm glass beads. The crude extracts were then assayed according to methods readily available to the skilled artisan.

The produced alcohol compounds were quantified by a gas chromatograph (GC) equipped with flame ionization detector. The system consisted of model 5890A GC (Hewlett Packard, Avondale, Pa.) and a model 7673A automatic injector, sampler and controller (Hewlett Packard). The separation of alcohol compounds was carried out by A DB-WAX capillary column (30 m, 0.32 mm-i.d., 0.50 μm-film thickness) purchased from Agilent Technologies (Santa Clara, Calif.). GC oven temperature was initially held at 40° C. for 5 min and raised with a gradient of 15° C./min until 120° C. And then it was raised with a gradient 50° C./min until 230° C. and held for 4 min. Helium was used as the carrier gas with 9.3 psi inlet pressure. The injector and detector were maintained at 225° C. 0.5 ul supernatant of culture broth was injected in split injection mode (1:15 split ratio). Isobutanol was used as the internal standard.

For other secreted metabolites, filtered supernatant was applied (20 ul) to an Agilent 1100 HPLC equipped with an auto-sampler (Agilent Technologies) and a BioRad (Biorad Laboratories, Hercules, Calif.) Aminex HPX87 column (0.5 mM H2SO4, 0.6 ml/min, column temperature at 65° C.). Glucose was detected with a refractive index detector, while organic acids were detected using a photodiode array detector at 210 nm. Concentrations were determined by extrapolation from standard curves.

Expression of C. acetobutylicum pathway in E. coli leads to 1-butanol production. To produce 1-butanol in E. coli, a set of genes for 1-butanol production (FIG. 1) were transferred into E. coli host cells. These genes (thl, hbd, crt, bcd, etfAB, adhE2) were cloned and expressed in E. coli using two plasmids (pJCL50 and pJCL60, see Table 1) under the control of the IPTG inducible P_{L}lacO1 promoter. The activity of these gene products were detected by enzyme assaysm, except bcd and etfAB which code for butyryl-CoA dehydrogenase (Bcd) and an electron transfer flavoprotein (Etf). The activity of butyryl-CoA dehydrogenase was not conclusively demonstrated using crude extract from cells that expressed bcd and etfAB. This difficulty was possibly due to the instability of the enzyme.

Despite the inconclusive demonstration of Bcd activity, the expression of this synthetic pathway produced 13.9 mg/L of 1-butanol under anaerobic conditions (FIG. 27A). In contrast to the suspected oxygen sensitivity, a slight increase in the oxygen level increased the production of 1-butanol, suggesting that the NADH produced anaerobically was insufficient to supply for 1-butanol production. In a completely aerobic condition, on the other hand, E. coli consumes both acetyl-CoA and NADH in TCA cycle and respiration, and thus likely contributes to the decreased 1-butanol production (FIG. 27).

In addition to the C. acetobutylicum thiolase (coded by thl), acetyl-CoA acetyltranserase from E. coli (coded by atoB) was overexpressed to examine its ability to catalyze the reaction of acetyl-CoA to acetoacetyl-CoA. Interestingly, the production of 1-butanol increased more than three-fold (FIG. 27), possibly because of the higher activity of this native enzyme. To determine whether homologues and isoenzymes of Bcd from other organisms would be more effective in E. coli, bcd and etfAB from M. elsdenii and ccr from S. coelicolor, which encodes a crotonyl-CoA reductase (Ccr) (that does not require an Etf for activity), were expressed in place of their counterparts from C. acetobutylicum. The activity of S. coelicolor Ccr, but not M. elsdenii Bcd, was detected conclusively by enzyme assays using crude extracts. However, the M. elsdenii and S. coelicolor genes led to lower production of 1-butanol in E. coli (FIG. 27B). Nevertheless, alternative genes from other organisms can improve 1-butanol production in E. coli. The use of a user-friendly host facilitates such exploration.

To further improve 1-butanol production, deletion of host pathways that compete with the 1-butanol pathway for acetyl-CoA and NADH was performed. FIG. 27C shows that deletion of ldhA, adhE, and frdBC from WT, complete with the 1-butanol production pathway (JCL184), doubled the production of 1-butanol by significantly reducing the amount of lactate, ethanol, and succinate produced (Table 4), consistent with the result shown for pyruvate production. The decision to knock out the native adhE in E. coli and replace it with adhE2 from C. acetobutylicum was based on the relative affinities of each ADH enzyme towards acetyl-CoA and butyryl-CoA (Table 4). While the activity of the E. coli ADH towards butyryl-CoA is not much less than the C. acetobutylicum ADH, its activity torwards acetyl-CoA is four times higher than the C. acetobutylicum ADH for the same substrate. This ratio favors adhE2 over adhE for 1-butanol production.

TABLE 4
Metabolic Byproducts of 1-Butanol Producing Strains

| Knockout genes | | | | | | | Product concentrations (mM) | | | | | | |
|---|---|---|---|---|---|---|---|---|---|---|---|---|---|
| adh | ldh | frd | fnr | pta | pfl | Butanol | Acetate | Ethanol | Formate | Pyruvate | Lactate | Succinate | Glucose¹ |
| | | | | | | 1.9 | 13.5 | 15.2 | 19.5 | 2.1 | 41.8 | 3.4 | 44.9 |
| Δ | Δ | Δ | | | | 3.7 | 15.2 | 6.0 | 23.1 | 4.0 | 5.4 | 0.7 | 30.7 |
| Δ | Δ | Δ | Δ | | | 2.1 | 11.8 | 5.0 | 16.4 | 2.4 | 2.5 | 1.2 | 22.2 |
| Δ | Δ | Δ | | Δ | | 2.7 | 1.3 | 3.0 | 18.5 | 12.7 | 2.4 | 1.1 | 28.2 |
| Δ | Δ | Δ | Δ | Δ | | 5.0 | 1.5 | 15.5 | 21.0 | 23.4 | 3.0 | 1.7 | 42.8 |
| Δ | Δ | Δ | Δ | | Δ | 0.1 | 4.9 | 1.0 | 3.5 | 6.0 | 2.9 | 2.5 | 14.1 |
| Δ | Δ | Δ | | Δ | Δ | 0.1 | 0.7 | 0.5 | 2.1 | 10.9 | 1.9 | 1.2 | 14.3 |
| Δ | Δ | Δ | Δ | Δ | Δ | 0.2 | 0.7 | 1.7 | 3.0 | 11.8 | 2.9 | 2.3 | 18.2 |

Cells were grown semi-aerobically in M9 media with the addition of 0.1% casamino acids at 37° C. for 24 hr.
¹Glucose Consumed

Although the deletions in JCL184 (ΔldhA, ΔadhE, ΔfrdBC) resulted in the decrease of most fermentation products, a significant amount of acetate was produced. To further increase 1-butanol production, pta was deleted. While acetate production was decreased considerably, JCL275 (ΔldhA, ΔadhE, ΔfrdBC, Δpta) led to a lower production of 1-butanol.

The deletion of pflB nearly abolished 1-butanol production, indicating that pyruvate-formate lyase (Pfl) was an enzyme responsible for the production of acetyl-CoA from pyruvate under the experimental condition (FIG. 27C). The use of Pfl to produce acetyl-CoA rather than the pyruvate dehydrogenase complex (PDHc) suggests that the condition does not provide enough NADH to fully reduce glucose to 1-butanol. This is supported by the data in FIG. 27A which shows that allowing a small amount of oxygen during growth, and thus elevating the activity of PDHc, increases the amount of 1-butanol produced compared to a completely anaerobic condition. This strain also produces a large amount of pyruvate due to insufficient NADH to make 1-butanol and the host's inability to produce lactate or acetate. It is therefore desirable to activate PDHc for the production of 1-butanol, since the reducing power is stored in NADH rather than formate. To achieve elevated expression of PDHc, the fnr gene, an anaerobic regulator that represses the expression of PDHC genes during anaerobic growth, was deleted. The deletion of fnr from the host decreased 1-butanol production. However, when both pta and fnr were deleted, production of 1-butanol improved nearly three-fold over wild type levels (about 373 mg/L). This improvement in 1-butanol production was accompanied by an increase of ethanol production to wild type levels, as well as a further increase in the secretion of pyruvate.

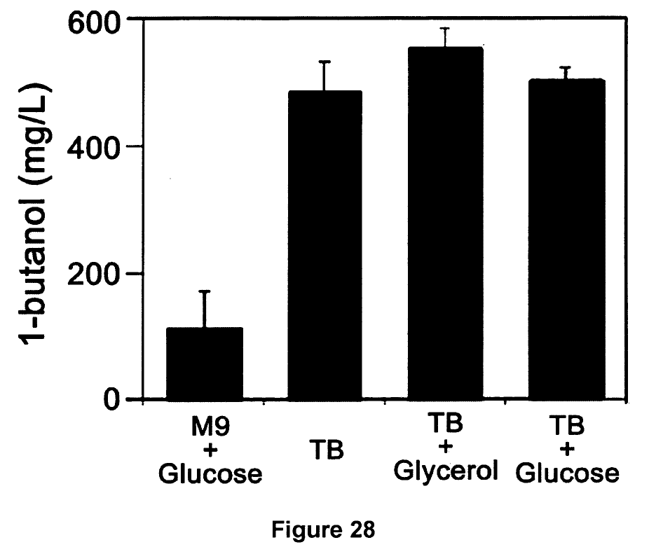

Various growth media were examined to increase the titer of 1-butanol. JCL187 (ΔadhE, ΔldhA, ΔfrdBC, Δfnr, Δpta containing pJCL17 and pJCL60) was grown in rich media (TB) supplemented with different carbon sources as well as minimal media for comparison. FIG. 28 shows that growth in rich media increased 1-butanol production, as cultures in TB supplemented with glycerol produced fivefold more 1-butanol (552 mg/L) than cultures grown in M9 (113 mg/L).

Additionally, the data demonstrate that E. coli can tolerate 1-butanol up to a concentration of 1.5% (data not shown), which is similar to published results found for the native producer C. acetobutylicum (Lin and Blaschek, 1983). As 1-butanol production in E. coli is optimized and product titers increase, improvement in the tolerance to 1-butanol can be achieved using similar strategies that have resulted in ethanol tolerant mutants.

It is to be understood that the inventions are not limited to particular compositions or biological systems, which can, of course, vary. It is also to be understood that the terminology used herein is for the purpose of describing particular embodiments only, and is not intended to be limiting.

The examples set forth above are provided to give those of ordinary skill in the art a complete disclosure and description of how to make and use the embodiments of the devices, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

What is claimed is:

1. A recombinant microorganism comprising a biochemical pathway to produce n-butanol from fermentation of a suitable carbon substrate the biochemical pathway comprising an acetoacetyl-coA intermediate, wherein the biochemical pathway comprises at least one heterologous polypeptide compared to a corresponding parental microorganism.

2. The recombinant microorganism of claim 2, comprising elevated expression of a polypeptide having keto thiolase activity, as compared to a parental microorganism, wherein the recombinant microorganism produces a metabolite comprising acetoacetyl-CoA from a substrate comprising acetyl-CoA.

3. The recombinant microorganism of claim 2, wherein the polypeptide having keto thiolase activity is encoded by a polynucleotide having at least about 50% identity to a sequence as set forth in SEQ ID NO:30, 66, 68, or 66 and 68.

4. The recombinant microorganism of claim 2, wherein the polypeptide having keto thiolase activity is encoded by an atoB gene or homolog thereof, or a fadA gene or homolog thereof.

5. The recombinant microorganism of claim 4, wherein the atoB gene or fadA gene is derived from the genus Escherichia.

6. The recombinant microorganism of claim 5, wherein the Escherichia is E. coli.

7. The recombinant microorganism of claim 1, comprising elevated expression of a polypeptide having acetyl-CoA acetyltransferase, as compared to a parental microorganism, wherein the recombinant microorganism produces a metabolite comprising acetoacetyl-CoA from a substrate comprising acetyl-CoA.

8. The recombinant microorganism of claim 7, wherein the polypeptide having acetyl-coA acetyltransferase activity is encoded by a polynucleotide having at least about 50% identity to a sequence as set forth in SEQ ID NO:32.

9. The recombinant microorganism of claim 7, wherein the polypeptide having acetyl-CoA acetyltransferase activity is encoded by a thl gene or homolog thereof.

10. The recombinant microorganism of claim 9, wherein the thl gene is derived from the genus Clostridium.

11. The recombinant microorganism of claim 9, wherein the Clostridium is C. acetobutylicum.

12. The recombinant microorganism of claim 1, comprising elevated expression of a polypeptide having hydroxybutyryl-CoA dehydrogenase activity, as compared to a parental microorganism, wherein the recombinant microorganism produces a metabolite comprising 3-hydroxybutyryl-CoA from a substrate comprising acetoacetyl-CoA.

13. The recombinant microorganism of claim 12, wherein the polypeptide having hydroxybutyryl-CoA activity is encoded by a polynucleotide having at least about 50% identity to a sequence as set forth in SEQ ID NO:36.

14. The recombinant microorganism of claim 12, wherein the hydroxybutyryl-CoA dehydrogenase is encoded by an hbd gene or homolog thereof.

15. The recombinant microorganism of claim 14, wherein the hbd gene is derived from a microorganism selected from the group consisting of Clostridium acetobutylicum, Clostridium difficile, Dastricha ruminatium, Butyrivibrio fibrisolvens, Treponema phagedemes, Acidaminococcus fermentans, Clostridium kluyveri, Syntrophosphora bryanti, and Thermoanaerobacterium thermosaccharolyticum.

16. The recombinant microorganism of claim 15, wherein the microorganism is Clostridium acetobutylicum.

17. The recombinant microorganism of claim 1, comprising elevated expression of a polypeptide having crotonase activity, as compared to a parental microorganism, wherein the recombinant microorganism produces a metabolite comprising crotonyl-CoA from a substrate comprising 3-hydroxybutyryl-CoA.

18. The recombinant microorganism of claim 17, wherein the polypeptide having crotonase activity is encoded by a polynucleotide having at least about 50% identity to a sequence as set forth in SEQ ID NO:34.

19. The recombinant microorganism of claim 17, wherein the crotonase is encoded by a crt gene or homolog thereof.

20. The recombinant microorganism of claim 19, wherein the crt gene is derived from a microorganism selected from the group consisting of Clostridium acetobutylicum, Butyrivibrio fibrisolvens, Thermoanaerobacterium thermosaccharolyticum, and Clostridium difficile.

21. The recombinant microorganism of claim 20, wherein the microorganism is Clostridium acetobutylicum.

22. The recombinant microorganism of claim 1, comprising elevated expression of a polypeptide having crotonyl-CoA reductase, as compared to a parental microorganism, wherein the recombinant microorganism produces a metabolite comprising butyryl-CoA from a substrate comprising crotonyl-CoA.

23. The recombinant microorganism of claim 22, wherein the polypeptide having crotonyl-coA reductase activity is encoded by a polynucleotide having at least about 50% identity to a sequence as set forth in any one of SEQ ID NOs:50, 52, 54, 56, 58, 60 and 62.

24. The recombinant microorganism of claim 23, wherein the polypeptide having crotonyl-CoA reductase is encoded by a ccr gene or homolog thereof.

25. The recombinant microorganism of claim 24, wherein the ccr gene is derived from the genus Streptomyces.

26. The recombinant microorganism of claim 25, wherein the Streptomyces is S. coelicolor or S. collinus.

27. The recombinant microorganism of claim 1, comprising elevated expression of a polypeptide having butyryl-CoA dehydrogenase, as compared to a parental microorganism, wherein the recombinant microorganism produces a metabolite comprising butyryl-CoA from a substrate comprising crotonyl-CoA.

28. The recombinant microorganism of claim 27, wherein the polypeptide having butyryl-CoA dehydrogenase activity is encoded by a polynucleotide having at least about 50% identity to a sequence as set forth in SEQ ID NO:38 or 44.

29. The recombinant microorganism of claim 27, wherein the polypeptide having butyryl-CoA dehydrogenase activity is encoded by a bcd gene or homolog thereof.

30. The recombinant microorganism of claim 29, wherein the bcd gene is derived from Clostridium acetobutylicum, Mycobacterium tuberculosis, or Megasphaera elsdenii.

31. The recombinant microorganism of claim 1, comprising elevated expression of a polypeptide having aldehyde/alcohol dehydrogenase activity, as compared to a parental microorganism, wherein the recombinant microorganism produces a metabolite comprising buteraldehyde from a substrate comprising butyryl-CoA.

32. The recombinant microorganism of claim 31, wherein the polypeptide having aldehyde/alcohol dehydrogenase activity is encoded by a polynucleotide having at least about 50% identity to a sequence as set forth in SEQ ID NO:64.

33. The recombinant microorganism of claim 31, wherein the polypeptide having aldehyde/alcohol dehydrogenase is encoded by an aad gene or homolog thereof, or an adhE2 gene or homolog thereof.

34. The recombinant microorganism of claim 33, wherein the aad gene or adhE2 gene is derived from Clostridium acetobutylicum.

35. The recombinant microorganism of claim 1, wherein the suitable carbon substrate comprises glucose.

36. The recombinant microorganism of claim 1, wherein the recombinant microorganism comprises one or more deletions or knockouts in a gene encoding an enzyme that catalyzes the conversion of acetyl-coA to ethanol, catalyzes the conversion of pyruvate to lactate, catalyzes the conversion of fumarate to succinate, catalyzes the conversion of acetyl-coA and phosphate to coA and acetyl phosphate, catalyzes the conversion of acetyl-coA and formate to coA and pyruvate, or condensation of the acetyl group of acetyl-CoA with 3-methyl-2-oxobutanoate (2-oxoisovalerate).

37. The recombinant microorganism of claim 1, further comprising reduced ethanol dehydrogenase activity, lactate dehydrogenase activity, fumarate reductase activity, phosphate acetyltransferase activity, formate acetyltransferase activity or any combination thereof.

38. The recombinant microorganism of claim 36, wherein the knockout or disruption comprises a deletion or disruption selected from the group consisting of adhE, ldhA, frdBC, pta, fnr, any combination thereof, any homolog or naturally occurring variants thereof.

39. The recombinant microorganism of claim 36, comprising the deletion or disruption of adhE, ldhA, frdBC, and pta, homologs or variants thereof.

40. The recombinant microorganism of claim 36, comprising the deletion or disruption of adhE, ldhA, frdBC, pta, and fnr, homologs or variants thereof.

41. The recombinant microorganism of claim 36, comprising the deletion or disruption of adhE, ldhA, frdBC, and fnr, homologs or variants thereof.

42. The recombinant microorganism of claim 1 or 36, further comprising reduced expression of an oxygen dependent transcription regulator.

43. The recombinant microorganism of claim 36, wherein the microorganism comprises a reduction or inhibition in the conversion of acetyl-coA to ethanol.

44. The recombinant microorganism of claim 36, wherein the recombinant microorganism comprises a reduction of an ethanol dehydrogenase thereby providing a reduced ethanol production capability.

45. The recombinant microorganism of claim 44, wherein the microorganism is derived from E. coli.

46. The recombinant microorganism of claim 45, wherein the ethanol dehydrogenase is an adhE, homolog or variant thereof.

47. The recombinant microorganism of claim 46, wherein the microorganism comprises a deletion or knockout of an adhE, homolog or variant thereof.

48. The recombinant micoorganism of claim 1, comprising a deletion or knockout selected from the group consisting of ΔadhE, ΔldhA, Δpta, ΔfrdB, ΔfrdC, ΔfrdBC, Δfnr, Δpta, Δpf1B and any combination thereof and comprising an expression or increased expression of an atoB, thl, adhE2, hbd, crt, bcd, ccr, and any combination thereof.

49. A recombinant microorganism comprising a recombinant biochemical pathway to produce n-butanol from fermentation of a suitable carbon substrate, wherein the recombinant biochemical pathway comprises elevated expression of:
a) a keto thiolase as compared to a parental microorganism or an acetyl-CoA acetyltransferase as compared to a parental microorganism;
b) a hydroxybutyryl-CoA dehydrogenase as compared to a parental microorganism;
c) a crotonase as compared to a parental microorganism;
d) a crotonyl-CoA reductase as compared to a parental microorganism or a butyryl-CoA dehydrogenase as compared to a parental microorganism; and
e) an alcohol dehydrogenase (ADH) as compared to a parental microorganism.

50. The recombinant microorganism of claim 49, wherein the suitable carbon substrate comprises glucose.

51. A method of producing a recombinant microorganism that converts a suitable carbon substrate to n-butanol, the method comprising transforming a microorganism with one or more polynucleotides encoding polypeptides having keto thiolase or acetyl-CoA acetyltransferase activity, hydroxybutyryl-CoA dehydrogenase activity, crotonase activity, crotonyl-CoA reductase or butyryl-CoA dehydrogenase, activity, and alcohol dehydrogenase activity.

52. The method of claim 51, wherein the suitable carbon substrate comprises glucose.

53. A method for producing n-butanol, the method comprising inducing over-expression of an atoB gene, an hbd and crt genes, a ccr gene, or an adhE2 gene, or any combination thereof, in an organism, wherein the organism produces n-butanol when cultured in the presence of a suitable carbon substrate.

54. A method for producing n-butanol, the method comprising:
(i) inducing over-expression of a thl gene in an organism;
(ii) inducing over-expression of an hbd and crt genes in an organism;
(iii) inducing over-expression of a bcd gene in the organism; and
(iv) inducing over-expression of an adhE2 gene in the organism; or
(v) inducing over-expression of (i), (ii), (iii), and (iv).

55. The method of claim 53 or claim 54, wherein the suitable carbon substrate comprises glucose.

56. A recombinant vector comprising:
(i) a first polynucleotide encoding a first polypeptide that catalyzes the conversion of acetoacetyl-coA to 3-hydroxybutyryl-CoA;
(iii) a second polynucleotide encoding a second polypeptide the catalyzes the conversion of 3-hydroxybutyryl-CoA to crotonyl-CoA; and
(iv) a third polynucleotide encoding a third polypeptide that catalyzes the reduction of crotonyl-CoA to butyryl-CoA.

57. The recombinant vector of claim 56, wherein the first polynucleotide encodes a 3-hydroxybutyryl-CoA dehydrogenase.

58. The recombinant vector of claim 57, wherein the 3-hydroxybutyryl-CoA dehydrogenase is encoded by a polynucleotide having at least 50%, 60%, 70%, 80%, 90%, 95%, 98% or 99% identity to a hbd gene.

59. The recombinant vector of claim 58, wherein the hbd gene comprises a C. acetobutylicum hbd gene.

60. The recombinant vector of claim 56, wherein the second polynucleotide encodes a crotonase.

61. The recombinant vector of claim 60, wherein the crotonase is encoded by a polynucleotide having at least 50%, 60%, 70%, 80%, 90%, 95%, 98% or 99% identity to a crt gene.

62. The recombinant vector of claim 61, wherein the crt gene comprises a C. acetobutylicum crt gene.

63. The recombinant vector of claim 56, wherein the third polynucleotide encodes a butyryl-CoA dehydrogenase complex.

64. The recombinant vector of claim 63, wherein the butyryl-CoA dehydrogenase complex is encoded by a polynucleotide having at least 50%, 60%, 70%, 80%, 90%, 95%, 98% or 99% identity to a bcd/etfA, bcd/etfB or bcd/etfAB gene.

65. The recombinant vector of claim 64, wherein the bcd/etfA, bcd/etfB or bcd/etfAB gene comprises a C. acetobutylicum or M. elsdenii bcd/etfA, bcd/etfB or bcd/etfAB gene.

66. The recombinant vector of claim 56, transfected into an E. coli overexpressing atoB.

67. The recombinant vector of claim 56, further comprising a fourth polynucleotide encoding a polypeptide that catalyzes the conversion of 2 acetyl-coA molecules to acetoacetyl-coA.

68. The recombinant vector of claim 67, wherein the fourth polynucleotide encodes an acetoacetyl-coA thiolase.

69. The recombinant vector of claim 68, wherein the acetoacetyl-coA thiolase is encoded by a polynucleotide having at least 50%, 60%, 70%, 80%, 90%, 95%, 98% or 99% identity to a thl gene.

70. The recombinant vector of claim 69, wherein the thl gene comprises a C. acetobutylicum thl gene.

71. The recombinant vector of claim 67, transfected into an E. coli.

72. The recombinant vector of claim 56 or 67, further comprising a polynucleotide encoding an aldehyde/alcohol dehydrogease that catalyzes the conversion of buytryl-coA to Butyraldehyde and 1-butanol.

73. The recombinant vector of claim 72, wherein the aldehyde/alcohol dehydrogease is encoded by a polynucleotide having at least 50%, 60%, 70%, 80%, 90%, 95%, 98% or 99% identity to an adhE2 gene.

74. The recombinant vector of claim 73, wherein the adhE2 gene comprises a C. acetobutylicum adhE2 gene.

75. The recombinant vector of claim 56 or 67, wherein the vector is a plasmid.

76. The recombinant vector of claim 56 or 67, wherein the vector is an expression vector.

77. The recombinant vector of claim 67, wherein the vector is a plasmid.

78. The recombinant vector of claim 67, wherein the vector is an expression vector.

79. A recombinant host cell comprising the expression vector of claim 76.

80. A recombinant host cell comprising the expression vector of claim 78.

81. The recombinant host cell of claim 80, wherein the recombinant host cell expresses thl, hbd, crt, bcd, etfAB, and adhE2 genes.
